(19) United States
(12) Patent Application Publication
(10) Pub. No.: US 2002/0132832 A1
(43) Pub. Date: Sep. 19, 2002

(54) Treatment of erectile dysfunction

(76) Inventors: Thomas M. Mills, Augusta, GA (US); Christopher J. Wingard, Augusta, GA (US); R. Clinton Webb, Matinez, GA (US); Ronald W. Lewis, Augusta, GA (US); Kanchan Chitaley, Augusta, GA (US)

Correspondence Address: Cynthia B. Rothschild, Esq., Kilpatrick Stockton LLP, 1001 W. 4th Street, Winston-Salem, NC, 27101, US

(21) Appl. No.: 10/040,010

(22) Filed: Jan. 4, 2002

Related U.S. Application Data
(60) Provisional application No. 60/260,062, filed on Jan. 5, 2001.
(60) Provisional application No. 60/267,296, filed on Feb. 8, 2001.

(51) Int. Cl. A61K 31/4409

(52) U.S. Cl. 514/352

(57) ABSTRACT

The invention relates to the treatment of sexual dysfunction by inhibition of vasoconstriction leading to the relaxation of smooth muscles in male or female erectile tissue. In one aspect, the invention comprises methods for treating male and female sexual dysfunction which comprises administering a composition comprising a compound which attenuates RhoA and/or Rho-kinase activity in an organ subject to sexual stimulation in a pharmaceutically acceptable carrier to an individual in need of such treatment. In one embodiment, the compound comprises an inhibitor of the enzyme Rho-kinase. In other embodiments, compounds targeted to other enzymes involved in phosphorylation of myosin light chain are used. Also described are compositions and kits for performing the methods of the invention.

This application claims the benefit of priority to U.S. Provisional Application Serial No. 60/260,062, filed Jan. 5, 2001, and U.S. Provisional Application Serial No. 60/267,296, filed Feb. 8, 2001. The entire disclosure of both applications is hereby incorporated by reference herein.

The United States Government may have rights in this invention which was supported by National Institutes of Health research grants: NIH HL-18575, entitled “Metabolic Determinants of Hypertension” to R. Clinton Webb, and NIH DK-59467, entitled “Erectile Function and the Influence of Rho-kinase” to Christopher J. Wingard.

### FIELD OF THE INVENTION

The invention relates to the treatment of male and female sexual dysfunction. In particular, the invention relates to methods and compositions for inducing relaxation of smooth muscle cells through the inhibition of endogenous vasoconstriction by attenuation of the RhoA/Rho-kinase pathway in the penile and clitoral vasculature.

### BACKGROUND OF THE INVENTION

The human sexual response in both males and females results from an interplay of physiological, psychological, and hormonal factors. One common aspect of the sexual response in males and females, however, is the vasoactive response, which results in engorgement of the sexual tissues of the genitalia with blood as a result of vascular smooth muscle relaxation in response to sexual stimulation. Thus, blood pressure and blood flow inside the penis and clitoris increase when smooth muscles of the pudental vasculature relax. This arterial influx of blood causes enlargement of the penile or clitoral corpora cavernosa and results in erection. In the penis, venous outflow is reduced by enlargement of the corpus cavernosum, permitting sustained high cavernosal blood pressure and maintained rigidity.

Relaxation of penile or clitoral smooth muscle and the accompanying vasodilation are triggered by the central nervous system and reinforced locally by reflex mechanisms. Most of the time, however, the body keeps the erectile tissue in a flaccid (non-erect) state by maintaining the smooth muscle tissues in the contracted state. Vasoconstrictors, such as norepinephrine (noradrenaline) and endothelin-1, help maintain the cavernosal smooth muscle tissue in a contracted state to keep blood flow low.

Impotence (erectile dysfunction in men) is generally defined as an inability to achieve and sustain an erection sufficient for satisfactory sexual performance and intercourse. Impotence can be due to psychological disturbances, neurological abnormalities, or other physiological disturbances including hormonal deficiencies or a combination of causes. Male impotence is estimated to affect 40% of men age 40 in the U.S., increasing with age to about 50% by 50 years, and is as high as 67% by the age of 70. In the United States, it is estimated that up to 30 million males may suffer from impotence.

Females can also have sexual dysfunction that increases with age and is associated with the onset of menopause and increased risk of vascular disorders. Thus, similar to men, sexual arousal in women is accompanied, at least in part, by increased blood flow which engorges the clitoris. Blood flow to the vagina also increases resulting in increased vaginal lubrication. Thus, female sexual dysfunction can result from an inability to attain or maintain vaginal lubrication and clitoral engorgement throughout the period of sexual activity (see e.g. Berman, J. R., et al., Eur. Urology 38, 20-29, 2000).

Previous work in the area of erectile dysfunction has focused on processes that result in smooth muscle relaxation. One mechanism which causes erection of the penis involves release of nitric oxide (NO), enabling relaxation of blood vessels in the cavernosal circulation during sexual stimulation. For example, the compound sildenafil (Viagra) is a type 5 phosphodiesterase inhibitor that potentiates the effects of local release of NO, thereby resulting in vascular smooth muscle relaxation. Studies have found sildenafil to have an overall 60% efficacy rate in the promotion of NO-mediated cavernosal vasorelaxation (Virag, R., Urology 54, 1073-77, 1999). Still, in those patients with severe erectile dysfunction (such as that resulting from diabetes or prostate surgery), sildenafil treatment was associated with a modest satisfaction rate (Jarow, I. P. et al., J. Urology, 102, 722-725, 1999). Moreover, only 30% of patents studied chose sildenafil treatment alone (Virag, R., 1999).

Other efforts to develop treatments for sexual dysfunction include U.S. Pat. No. 6,087,362, which describes treatment of sexual dysfunction by an oral therapy of administration of apomorphine and sildenafil which is directed to minimizing the side effects of each agent. Other patents describing treatments for sexual dysfunction include U.S. Pat. Nos. 6,166,061, 6,124,337, 6,100,286, 6,051,594, and 5,981,563 which describe oral formulations of the α-adrenergic antagonist, phentolamine. Also, U.S. Pat. No. 6,100,270 describes a method of treating male erectile dysfunction by administration of bicyclic heterocyclic pyrimidine compounds which are potent inhibitors of cyclic guanosine 3′,5′-monophosphate phosphodiesterases. In addition, U.S. Pat. No. 6,007,824 describes a composition and method for treating sexual dysfunction using a combination of L-arginine, ginseng, and Zizyphi fructus in an orally administered dosage.

Thus, there is an ongoing need for safe and reliable treatments for sexual dysfunction. Preferably, such treatments will target alternative biochemical pathways from those targeted by currently described treatment protocols utilizing vasodilators. A method which focuses on inhibition of vasoconstriction can target enzymes and biochemical pathways distinct from those targeted by vasodilators.

Currently, most vasodilators used to treat erectile dysfunction target signal transduction pathways that reduce calcium ion which is needed to initiate contractile activity in vascular smooth muscle. However, from a physiological standpoint, the initiation of vasoconstriction is a very acute event lasting only seconds to a few minutes. The erectile tissue is maintained in the flaccid state by long-term vasoconstriction through a signal transduction pathway that is calcium-independent. A signal pathway that maintains calcium-independent vasoconstriction is the RhoA/Rho-kinase pathway.

Thus, the present invention teaches inhibition of the RhoA/Rho-kinase pathway to treat erectile dysfunction. Development of alternate approaches to the treatment of sexual dysfunction should allow for combinations of drugs utilizing complementary mechanisms to be employed. By using low doses of complementary agents, side effects resulting from higher dosages of either agent alone are minimized, and the ability to treat sexual dysfunction due to a wide variety of causes is enhanced.

### SUMMARY OF THE INVENTION

The present invention describes compositions and methods for treating erectile dysfunction that provide a unique protocol which can be used on its own, or in addition to currently employed methods. The methods and compound of the present invention inhibit vasoconstriction in the sexual organs, thereby promoting an increase in blood flow. This increased blood flow allows for the tissue to be maintained in an erect (or engorged) state as required for satisfactory sexual performance.

In one aspect, the present invention comprises a method for treating male or female sexual dysfunction which comprises administering a composition comprising a compound which attenuates RhoA and/or Rho-kinase activity in an organ subject to sexual stimulation and a pharmaceutically acceptable carrier to an individual in need of such treatment.

In another aspect, the present invention comprises a composition for treating male or female sexual dysfunction comprising a compound which attenuates RhoA and/or Rho kinase activity in an organ subject to sexual stimulation and a pharmaceutically acceptable carrier.

In yet another aspect, the present invention comprises a kit for treating male or female sexual dysfunction comprising at least one compound which attenuates RhoA and/or Rho-kinase activity in an organ subject to sexual stimulation and a pharmaceutically acceptable carrier.

The foregoing focuses on the more important features of the invention in order that the detailed description which follows may be better understood and in order that the present contribution to the art may be better appreciated. There are, of course, additional features of the invention which will be described hereinafter and which will form the subject matter of the claims appended hereto. It is to be understood that the invention is not limited in its application to the specific details as set forth in the following description and figures. The invention is capable of other embodiments and of being practiced or carried out in various ways.

From the foregoing summary, it is apparent that an object of the present invention is to provide methods and compositions for treating male and female sexual dysfunction. These, together with other objects of the present invention, along with various features of novelty which characterize the invention, are pointed out with particularity in the following description and claims.

### BRIEF DESCRIPTION OF THE DRAWINGS

Various features, aspects, and advantages of the present invention will become more apparent with reference to the following description and accompanying drawings.

FIG. 1 illustrates the structure of Y-27632 as Formula I in accordance with an embodiment of the present invention.

FIG. 2 illustrates proposed pathways by which inhibitors of vasoconstriction act to prevent erectile dysfunction and the likely site of nitric oxide inhibition of the RhoA/Rho-kinase pathway during normal erection in accordance with an embodiment of the present invention.

FIG. 3 shows the in vivo effect of the Rho-kinase inhibitor Y-27632 on the ratio of corpus cavernosum pressure (CCP) to mean arterial pressure (MAP) in the presence and absence of ganglionic nerve stimulation in accordance with an embodiment of the present invention wherein (A) shows a dose-dependent increase in CCP/MAP upon intracavernosal injection of Y-27632 (n=at least 4 per dose); and (B) shows a potentiation of a voltage-dependent increase in CCP/MAP upon ganglionic stimulation induced by Y-27262 (saline, ▪; Y-27632, □).

FIG. 4 shows the effect of nitric oxide synthase (NOS) inhibition on Y-27632-induced potentiation of ganglionic-stimulated increases in the ratio of corpus cavernosum pressure (CCP) to mean arterial pressure (MAP) in accordance with an embodiment of the present invention wherein (A) shows a tracing of experimental results from one rat; and (B) shows restoration by Y-27632 of N^{ω}-nitro-L-arginine methyl ester (L-NAME) inhibition of ganglionic-induced increase in CCP/MAP (n=4).

FIG. 5 shows the in vitro effect of the Rho-kinase inhibitor Y-27632 on isolated rat cavernosal tissue in accordance with an embodiment of the present invention wherein (A) shows a relaxation of cavernosal strips pre-constricted with phenylephrine (PE) induced by increasing concentrations of Y-27632 with (▪) and without ♦ N^{ω}-nitro-L-arginine methyl ester (L-NAME); and (B) the response of cavernosal tissue pre-constricted with PE (n=6) or potassium chloride (KCl) (n=6) to Y-27632 (1 μM) in the presence of 10 μM L-NAME (n=at least 3) or 10 μM methylene blue (n=4).

FIG. 6 shows the additive effects of a nitric oxide (NO) donor drug (NOR-1) and the Rho-kinase inhibitor (Y-27632) in promoting an increase in intracavernosal pressure/mean arterial pressure (ICP/MAP) in accordance with an embodiment of the present invention.

FIG. 7 shows the effect of Y-27632 in endothelium-denuded vascular tissue or endothelium-intact vascular tissue in accordance with an embodiment of the present invention wherein (A) shows the effect of 1 μmol/L Y-27632 (i.e. inhibition of phenylephrine (PE) induced contraction) in endothelium-denuded (etched bar) or endothelium-intact (solid bars) tissue treated with N^{ω}-nitro-L-arginine (L-NNA) or 1H-[1,2,4]oxadiozolo-[4,3-a]quinoxalin-1 (ODQ), as compared to untreated endothelium intact tissue; (B) shows that treatment with 1, 10, or 100 μmol/L Y-27632 results in a concentration-dependent inhibition of the PE-induced contraction in endothelium-denuded rat vascular tissue; and (C) shows the effect of Rho-kinase inhibition on the relaxation response to the NO donor sodium nitroprusside (SNP) in endothelium-denuded vascular tissue.

FIG. 8 shows the erectile response to ganglionic stimulation (5 volts) upon administration of 10 μg/kg methoxamine (METHOX), 50 nmol Y-27632, and Y-27632 prior to methoxamine (Y+METHOX) as compared to a control (CONT) in accordance with an embodiment of the present invention.

FIG. 9 shows the erectile response to ganglionic stimulation (5 volts) upon administration of 5 pmol endothelin-1 (ET-1), 50 nmol Y-27632, and Y-27632 prior to ET-1 (Y+ET-1) as compared to a control (CONT) in accordance with an embodiment of the present invention.

FIG. 10 shows that treatment with Y-27632 returns the erectile response to near normal levels in hypertensive rats in accordance with an embodiment of the present invention, wherein (A) shows the erectile response (ganglionic-mediated increases in ICP/MAP) for sham (control) and deoxycortisone-salt (DOCA) induced hypertensive rats; (B) shows the erectile response (ganglionic-mediated increases in ICP/MAP) for control and spontaneously hypertensive rats which are stroke prone (SHRSP); (C) shows the erectile response (ganglionic-mediated increases in ICP/MAP) results for sham (control) and deoxycortisone-salt (DOCA) induced hypertensive rats after treatment with Y-27632; and (D) shows the erectile response (ganglionic-mediated increases in ICP/MAP) results for control and spontaneously hypertensive rats which are stroke prone (SHRSP) after treatment with Y-27632.

FIG. 11 shows the voltage dependent erectile responses of intact and castrate rats before and after Rho-kinase inhibition in accordance with an embodiment of the present invention, wherein (A) shows the tracings for one castrate rat and one intact rat for voltages (1-5 V) indicated above tracings, before and after administration of Y-27632; and (B) shows the mean erectile responses (after 2 min stimulation at indicated voltage) for several (N=5-6) animals (castrate or intact) for each group before and after administration of Y-27632.

### DETAILED DESCRIPTION OF THE INVENTION

In one aspect, the present invention comprises a method for treating male or female sexual dysfunction which comprises administering a composition comprising a compound which attenuates RhoA and/or Rho-kinase activity in an organ subject to sexual stimulation and a pharmaceutically acceptable carrier to an individual in need of such treatment.

In an embodiment, the composition comprises a compound that inhibits the activity of Rho-kinase enzyme in an organ subject to sexual stimulation. Preferably, the Rho-kinase inhibitor comprises the structural formula (I), or a functional derivative thereof,

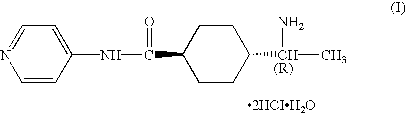

wherein a functional derivative comprises a compound which can inhibit the activity of Rho-kinase mediated phosphorylation and thereby increase intracavernosal pressure (ICP) (or corpus cavernosum blood pressure, CCP) relative to mean arterial pressure (MAP).

Thus, in an embodiment, the Rho-kinase inhibitor is administered in a dose that results in a increase in corpus cavernosum blood pressure (CCP) or intracavernosal pressure (ICP) (i.e. CCP and ICP are equivalent) relative to the mean arterial pressure (MAP). By measuring ICP (or CCP) relative to MAP, the extent of local vascular relaxation and engorgement of the cavernosal tissue is quantified. Hence, ICP/MAP (or CCP/MAP) is a measurement of the erectile response.

Preferably, the dose of the Rho-kinase inhibitor ranges from 2.0 to 400 nmol/kg body weight. More preferably, the dose ranges from 5.0 to 200 nmol/kg body weight. Even more preferably, the dose ranges from 40 to 100 nmol/kg body weight.

In an embodiment, the composition comprises a compound that reduces the amount of active Rho-kinase enzyme. Thus, the composition may comprise a compound that inhibits the activity of the upstream enzyme RhoA. In an embodiment, the composition may comprise a compound that inhibits translocation of RhoA enzyme to the cellular membrane. In an embodiment, the composition comprises a compound that inhibits binding of GTP to RhoA enzyme. In an embodiment, the composition comprises an inhibitor of Rho-kinase and a compound that potentiates the effects of nitric oxide. In an embodiment, the composition may comprise a compound that acts on a downstream target of Rho-kinase such as myosin light chain phosphatase, calponin, myosin light chain, CPI-17, and others.

Various methods may be used to administer the compositions of the present invention, depending upon the severity of the symptoms or the preference of the user. Thus, administration may be intracavernous, or in alternate embodiments, topical, oral, or sublingual. In another embodiment, administration of the compound is to the nasal passages, as for example, by an aerosol. Administration may also be transurethral or transrectal, as by a suppository and the like. In yet another embodiment, administration of the compound is by ionophoresis or electroporation. In another embodiment, various proteins in the RhoA/Rho-kinase signal transduction pathway may be altered using somatic gene therapy approaches.

The methods of the present invention may be used to treat sexual dysfunction arising from a variety of causes. For example, in an embodiment, the method may be used to treat sexual dysfunction associated with hypogonadism and more particularly, wherein the hypogonadism is associated with reduced levels of androgen hormones. In another embodiment, the method may be used to treat sexual dysfunction associated with a variety of causes including, but not limited to, hypertension, diabetes, or pelvic surgery. In addition, the method may be used to treat sexual dysfunction associated with treatment using certain drugs such as those to treat hypertension, depression or anxiety.

The present invention also provides the ability to treat diseases wherein the sexual dysfunction comprises an overly active erectile response, as for example the disorder of priapism. Thus, in one aspect, the present invention comprises a method to treat priapism in a patient comprising increasing the activity of the RhoA/Rho-kinase pathway in an organ subject to sexual stimulation in the patient.

In another aspect, the present invention comprises a composition for treating male or female sexual dysfunction comprising a compound which attenuates RhoA and/or Rho-kinase activity in an organ subject to sexual stimulation and a pharmaceutically acceptable carrier.

Preferably, the composition comprises a compound that inhibits the activity of Rho-kinase enzyme in an organ subject to sexual stimulation. More preferably, the composition comprises a compound comprising the structural formula (I) or a functional derivative thereof,

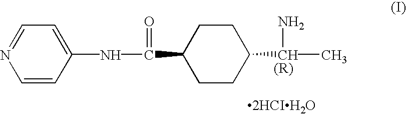

wherein a functional derivative comprises a compound which can inhibit the activity of Rho-kinase mediated phosphorylation and thereby increase intracavernosal pressure (ICP) (or corpus cavernosum blood pressure (CCP)) relative to mean arterial pressure MAP.

Thus, the composition of the present invention preferably comprises a dose of a Rho-kinase inhibitor that results in a increase in intracavernosal blood pressure (ICP) relative to the mean arterial pressure (MAP). Preferably, the dose of the Rho-kinase inhibitor ranges from 2.0 to 400 nmol/kg body weight. More preferably, the dose ranges from 5.0 to 200 nmol/kg body weight. Even more preferably, the dose ranges from 40 to 100 nmol/kg body weight.

In an embodiment, the composition comprises a compound that reduces the amount of active Rho-kinase enzyme. Thus, the composition may comprise a compound that inhibits the activity of the upstream enzyme RhoA. In an embodiment, the composition may comprise a compound that inhibits translocation of RhoA enzyme to the cellular membrane. In an embodiment, the composition comprises a compound that inhibits binding of GTP to RhoA enzyme. In an embodiment, the composition comprises an inhibitor of Rho-kinase and a compound that potentiates the effects of nitric oxide. In an embodiment, the composition may comprise a compound that acts on a downstream target of Rho-kinase, such as myosin light chain phosphatase, calponin, myosin light chain, CPI-17, and others.

In an embodiment, the composition of the invention is suitable for intracavernous injection. In another embodiment, the composition is suitable for transurethral administration. In another embodiment, the composition is suitable for topical application. In another embodiment, the composition is suitable for oral administration. In another embodiment, the compound is suitable for sublingual administration. In another embodiment, the compound is suitable for administration to the nasal passages as for example, by an aerosol. In another embodiment, the composition is suitable for transrectal application, as for example, by a suppository. In yet another embodiment, the composition is suitable for application by ionophoresis or electroporation.

In yet another aspect, the invention comprises a kit comprising a compound that attenuates RhoA/Rho-kinase activity in an organ subject to sexual stimulation and a pharmaceutically acceptable carrier. In an embodiment, the kit comprises aliquots packaged in units suitable for dispensing as individual dosages.

Preferably, the kit comprises a compound that inhibits the activity of Rho-kinase enzyme in an organ subject to sexual stimulation. More preferably, the kit comprises a compound comprising the structural formula (I), or a functional derivative thereof,

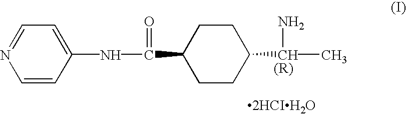

wherein a functional derivative comprises a compound which can inhibit the activity of Rho-kinase mediated phosphorylation and thereby increase intracavernosal pressure (ICP) (or corpus cavernosum blood pressure, CCP) relative to mean arterial pressure (MAP).

In an embodiment, the kit comprises individual doses of a Rho-kinase inhibitor that results in a increase in intracavernosal blood pressure (ICP) relative to the mean arterial pressure (MAP). Preferably, the dose of the Rho-kinase inhibitor ranges from 2.0 to 400 nmol/kg body weight. More preferably, the dose ranges from 5.0 to 200 nmol/kg body weight. Even more preferably, the dose ranges from 40 to 100 nmol/kg body weight.

In an embodiment, the kit comprises a compound that reduces the amount of active Rho-kinase enzyme. Thus, the kit may comprise a compound that inhibits the activity of the upstream enzyme RhoA. In an embodiment, the kit may comprise a compound that inhibits translocation of RhoA enzyme to the cellular membrane. In an embodiment, the kit comprises a compound that inhibits binding of GTP to RhoA enzyme. In an embodiment, the kit comprises a Rho-kinase inhibitor and a second compound that potentiates the effects of nitric oxide

In an embodiment, the units suitable for dispensing as individual doses comprise liquid solution for intracavernous injection packaged in single dosage vials. In an embodiment, the kit comprises units suitable for transurethral administration. In another embodiment, the units suitable for dispensing as individual doses comprise a topical cream or ointment. In another embodiment, the units suitable for dispensing as individual doses comprise a suppository for transrectal application. In another embodiment, the units suitable for dispensing as individual doses comprise a carrier for ionophoresis or electroporation. In yet another embodiment, the units suitable for dispensing as individual doses comprise tablets, lozenges or troches suitable for oral consumption or sublingual administration. In another embodiment, the units suitable for dispensing as individual doses are suitable for nasal administration, as for example, an aerosol with an appropriate spray dispenser.

Thus, the invention comprises methods and compositions for treating male or female sexual dysfunction. As used herein, sexual dysfunction includes erectile dysfunction in men, where erectile dysfunction is defined as the inability to achieve and maintain sufficient rigidity of the penis to permit penetration of the sexual partner during intercourse. In females, sexual dysfunction includes a failure of clitoral erection and/or a failure to attain (or maintain) sexually stimulated congestion of blood in the walls of the vagina, which results in inadequate vaginal lubrication. Thus, sexual dysfunction comprises both male and female sexual dysfunction which is due, at least in part, to lack of necessary blood flow in the erectile tissue of sexual organs.

Inhibition of Vasoconstriction

Smooth muscle relaxation leading to erection is thought to be mediated by the nitric oxide (NO)/cyclic GMP (cGMP) pathway, although other control pathways are involved as well (Andersson, K. E., Pharmacol Rev., 53, 417-450, 2001; Burnett, A. L., Biol. Reprod., 52, 485-489, 1995; Okamura., T., et al., Am J. Physiol. Heart Circ. Physiol., 274, H1075-H1081, 1998). NO is released from non-adrenergic/non-cholinergic nerves and endothelial cells to induce cavernosal smooth muscle relaxation. NO activates the enzyme guanylate cyclase, which then produces increased levels of cyclic guanosine monophosphate (cGMP), resulting in smooth muscle relaxation in the corpus cavernosum and allowing inflow of blood. Several drugs used to treat erectile dysfunction work by prolonging the effect of NO. For example, the compound sildenafil (Viagra) is a type 5 phosphodiesterase inhibitor used for the treatment of erectile dysfunction. Phosphodiesterase type 5 (PDE5) is responsible for degradation of cGMP in the corpus cavernosum. Thus, when sexual stimulation results in local release of NO, inhibition of PDE5 by sildenafil (Viagra) maintains increased levels of cGMP in the corpus cavernosum, resulting in vascular smooth muscle relaxation.

The present invention, however, targets inhibition of vasoconstrictors, and thereby provides an alternate approach for inducing smooth muscle relaxation unique from methods of treatment which focus on vasodilation of sexual organs. In the absence of an active NO/cGMP pathway, the arteriolar and sinusoidal smooth muscles remain in the contracted state, possibly mediated by the actions of norepinephrine (NE), endothelin-1 (ET-1) or other vasoconstrictors (Andersson, K. E., Pharmacol Rev., 53, 417-450, 2001; Dai, Y., et al., Am. J. Physiol. Regulatory Integrative Compl Physiol, 279, R25-R30, 2000; Feng et al., J. Biol Chem., 274, 3744-3752, 1999; Traish, A., Int. J. Impot. Res., 12, Suppl. 1, S48-63, 2000).

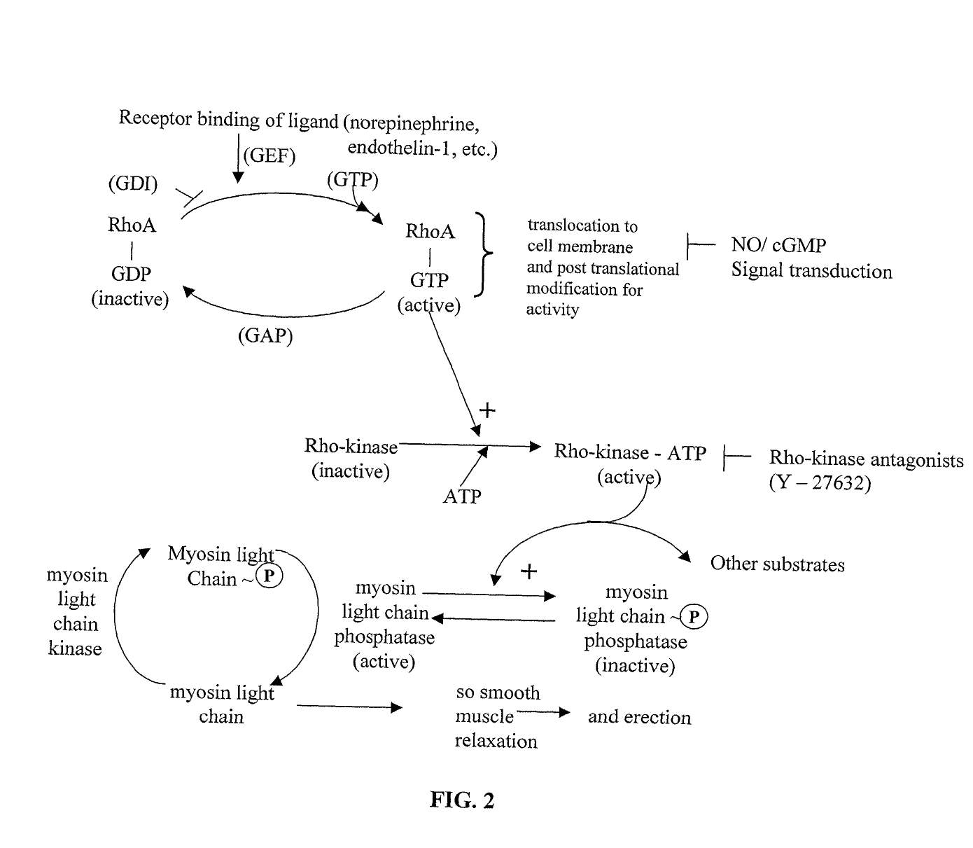

In one aspect, the present invention comprises attenuation of RhoA/Rho-kinase activity in an organ subject to sexual stimulation. For example, in an embodiment, and referring to FIG. 2, the invention comprises inhibition of an enzyme, Rho-kinase, as a method to inhibit vasoconstriction. Rho-kinase is an enzyme downstream of RhoA, and has been shown to promote contraction of fibroblast cells via the phosphorylation of myosin light chain (MLC) phosphatase (Somlyo, A. P. et al., Acta Physiol. Scand., 164, 437-448, 1998; Kimura, K., et al., Science 273, 245-248, 1997; Amano, M., et al., J. Biol. Chem., 271, 20246-20249, 1996; Kureishi, Y., et al., J. Biol. Chem., 272, 12257-12260, 1997; Feng, J. et al., J. Biol. Chem., 274, 3744-3752, 1999).

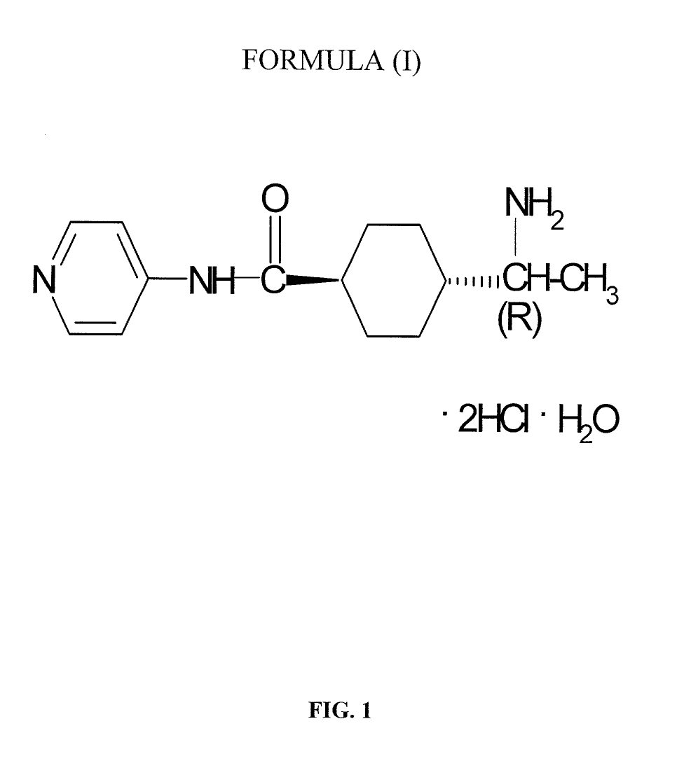

In an embodiment, the present invention comprises direct inhibition of Rho-kinase. Similar to other kinases, Rho-kinase uses the energy of the high energy phosphate of ATP for phosphorylation of its substrate (e.g. MLC phosphatase). In an embodiment, inhibition of Rho-kinase is due to an interference with ATP binding to Rho-kinase, thereby preventing donation of high energy phosphate moieties to the myosin light chain phosphatase. In an embodiment, the compound which binds to the Rho-kinase enzyme comprises a structure having the molecular formula C₁₄H₂₁N₃.2HCl.H₂O and the structural formula (I) shown in FIG. 1.

The compound shown as structure (I), Y-27632, comprises (+)-(R)-trans-4-(1-Aminoethyl)-N-(4-pyridyl)cyclohexanecarboxamide dihydrocholoride, monohydrate, and is manufactured by Mitsubishi Pharma Corporation (Osaka, Japan). Y-27632 has been shown to be 20 to 200 times more specific for Rho-kinase than Ca²⁺-dependent kinase (PKC), MLC kinase and cyclic AMP-dependent protein kinase (Uehata M., et al., Nature, 389, 990-994, 1997; Ishizaki T. et al., Mol. Pharmacol., 57, 976-983, 2000). In addition, Y-27632 has been shown to cause smooth muscle relaxation in cultured cells and in an animal model of hypertension (e.g. Uehata et al., Nature 389, 990-994, 1997; Ishizaki, T., et al., Mol. Pharmacol., 57, 976-983, 2000).

Thus, a functional Rho-kinase inhibitor comprises a compound that is able to inhibit the activity of Rho-kinase such that substrates for the enzyme are phosphorylated with reduced efficiency in the presence of the inhibitor. In an embodiment, this inhibition of the enzyme results in measurable smooth muscle relaxation. In erectile tissue, the smooth muscle relaxation may be detected (and quantified) as an increase in blood pressure in the corpus cavernosum (CCP or ICP) relative to the mean arterial pressure (MAP).

In another embodiment, the present invention comprises reducing the amount of active Rho-kinase enzyme. Thus, the invention may comprise inhibiting the activity of the upstream enzyme RhoA (FIG. 2). Like other members of the Rho family, RhoA serves as a cellular switch to regulate the activation of various signaling cascades such as the serine/threonine kinases: protein kinase N, the PAK family, and ROK/Rho-kinase, as well as adaptor proteins such as rophilin, rhotekin, and citron (Ridley, A., Curr. Biol., 6, 1256-1264, 1996; Sahai, E., et al., EMBO J., 17, 1350-1361, 1998; Apenstrom, P., Curr. Opin. Cell. Biol., 11, 95-102, 1999). In its guanosine diphosphate (GDP) bound form, RhoA is inactive, and is predominantly cytosolic. Guanosine nucleotide dissociation inhibitors (GDIs) bind cytosolic RhoA, inhibiting the release of GDP from RhoA, thus promoting its inactive state. Guanine nucleotide exchange factors (GEFs) promote the activation of RhoA by destabilizing bound GDP, thus enabling the binding of GTP to RhoA (FIG. 2) (Somlyo, A. P., et al., Acta Physiol. Scand., 164, 437-448, 1998; Somlyo et al., J. Physiol., 522, 177-185, 2000; Pfitzer, G., et al., Acta Physiol Scand., 164, 449-456, 1998). In an embodiment, the present invention comprises inhibiting binding of GTP to RhoA enzyme.

Translocation to the membrane, as well as additional post-translational modifications such as geranylgeranylation to promote membrane binding, are also components of the RhoA activation process. Thus, in an embodiment, the present invention may comprise inhibiting translocation of RhoA enzyme to the cellular membrane.

In an embodiment, the target of Rho-kinase is MLC phosphatase (FIG. 2). In the phosphorylated state, MLC phosphatase is inactive. With MLC phosphatase activity inhibited, the resulting high levels of phosphorylated myosin light chain keep the sexual organs in a nonerect state. For example, the phophatase inhibitor sodium orthovanadate (SOV) (Di Salvo, J. et al, Arch Biochem Biophys., 304, 386-391, 1993) inhibits the erectile response in a dose dependent manner when injected into the corpus cavernosum immediately before electrical stimulation of the autonomic ganglion, with significant inhibition seen at 0.1 and 1 μmol/L SOV (data not shown).

There are, however, other substrates for Rho-kinase phosphorylation, several of which are involved in smooth muscle relaxation and contraction (Table 1). Thus, in an embodiment, inhibition of Rho-kinase leads to a reduction in phosphorylation of myosin light chain (MLC) and decreased vasoconstriction. In another embodiment, inhibition of Rho-kinase leads to a reduction in phosphorylation of CPI-17 and decreased vasoconstriction. Alternatively, inhibition of Rho-kinase leads to a reduction in phosphorylation of calponin, and decreased vasoconstriction. In yet another embodiment, inhibition of Rho-kinase leads to a reduction in phosphorylation of adducin and decreased vasoconstriction.

TABLE 1

| | | Effect of Rho |
|---|---|---|
| Protein | Action | kinase inhibition |
| Myosin light chain phosphatase | Activity decreased when phosphorylated by | RELAXATION |
| | Rho-kinase | |
| Myosin light chain | When phosphorylated by Rho-kinase, activity | RELAXATION |
| | is increased promoting vasoconstriction | |
| CPI-17 | Inhibits MLC phosphatase; phosphorylation by | RELAXATION |
| | Rho-kinase increases its inhibitory effect | |
| | thereby promoting vasoconstriction | |
| Calponin | Binds to F-actin to inhibit actin-activated | RELAXATION |
| | myosin ATPase activity; phosphorylation by | |
| | Rho-kinase promotes vasoconstriction | |
| Adducin | Cytoskeletal protein associated with protein | RELAXATION |
| | transport; when phosphorylated by Rho- | |
| | kinase, may influence membrane polarization | |

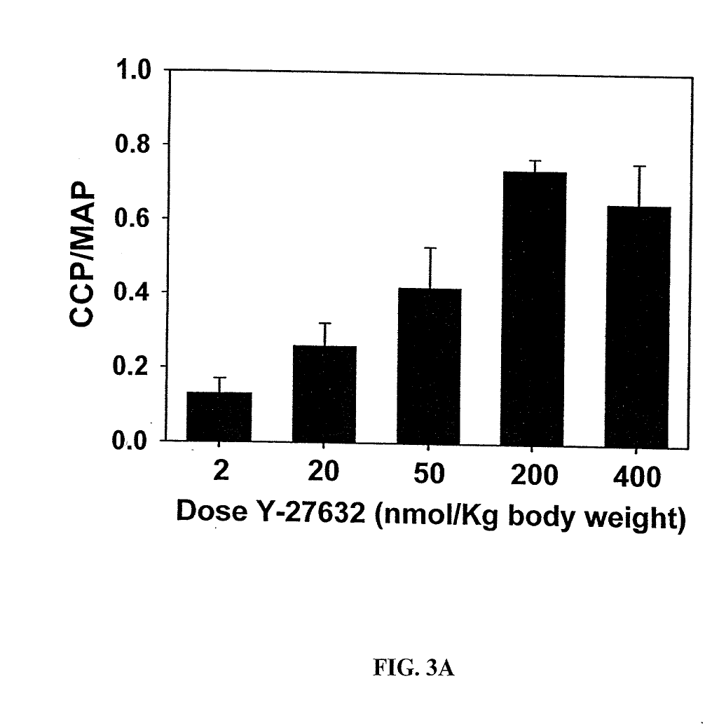

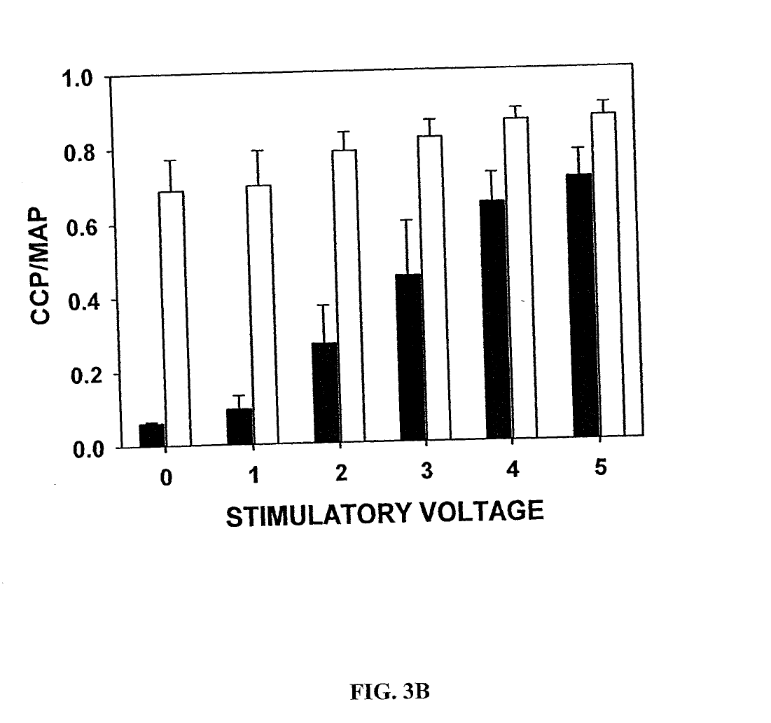

Thus, in an embodiment, the methods and compositions of the present invention exploit a Rho-kinase-mediated pathway which facilitates the maintenance of cavernosal vasoconstriction in the flaccid penis. Western blot data demonstrate the endogenous expression of Rho-kinase protein in rat cavernosal tissue (Chitaley, K., et al, Nature Med., 7, 119-122, 2001). Referring now to FIG. 3A, intracavernosal injection of Rho-kinase inhibitors such as Y-27632 causes a dose-dependent increase in the ratio of corpus cavernosum pressure (CCP) relative to mean arterial pressure (MAP), or CCP/MAP. By measuring CCP or ICP relative to MCP, the extent of local vascular relaxation and engorgement of the cavernosal tissue is quantified. The ability of Y-27632 to increase intracavernosal pressure independent of voltage stimulation indicates the presence of a high level of basal Rho-kinase activity in the cavernosal smooth muscle. In addition, administration of Y-27632 into the cavernous sinuses potentiates the CCP/MAP response to ganglionic stimulation at each voltage (FIG. 3B, open bars).

Preferably, there is a discrete dosage range which provides efficacy for an increase in CCP while not influencing MAP. In an embodiment, and referring again to FIG. 3A, only upon administration of 400 nmol/kg or more of Y-27632 was the MAP value significantly decreased. Injection of lower doses of Y-27632 (e.g. 2.0-200 nmol/kg) has no significant effect on MAP but enhances CCP in a dose-dependent manner. A change in MAP upon intracavernosal injection of Rho-kinase inhibitors such as Y-27632 may be due to the systemic actions of the compound at this high dose. Thus, the effect of lower doses of Y-27632 (2.0-200 nmol/kg) to increase CCP/MAP without significantly altering MAP supports a potential localized therapeutic use of Rho-kinase inhibitors in the treatment of erectile dysfunction.

NO Mediated Erection

Stimulation of the major pelvic ganglion may increase the CCP/MAP ratio by NO-mediated cellular pathways or other mechanisms (Reilly, C. M., et al., J. Andrology. 18, 110-115 (1997)). The Rho-kinase inhibitors of the present invention act by a pathway which may interact with, but is not dependent upon, the mediators nitric oxide and guanylate cyclase. For example, the Rho-kinase inhibitor Y-27632 can potentiate even maximal voltage-stimulated increases in CCP/MAP (FIG. 3B). The ability of Rho-kinase inhibitors to increase the erectile response above that seen with maximal ganglionic stimulation (i.e. NO-mediated) indicates the presence of a remaining vasoconstrictor component of the cavernosal tissue not subject to NO-mediated relaxation (FIG. 3B).

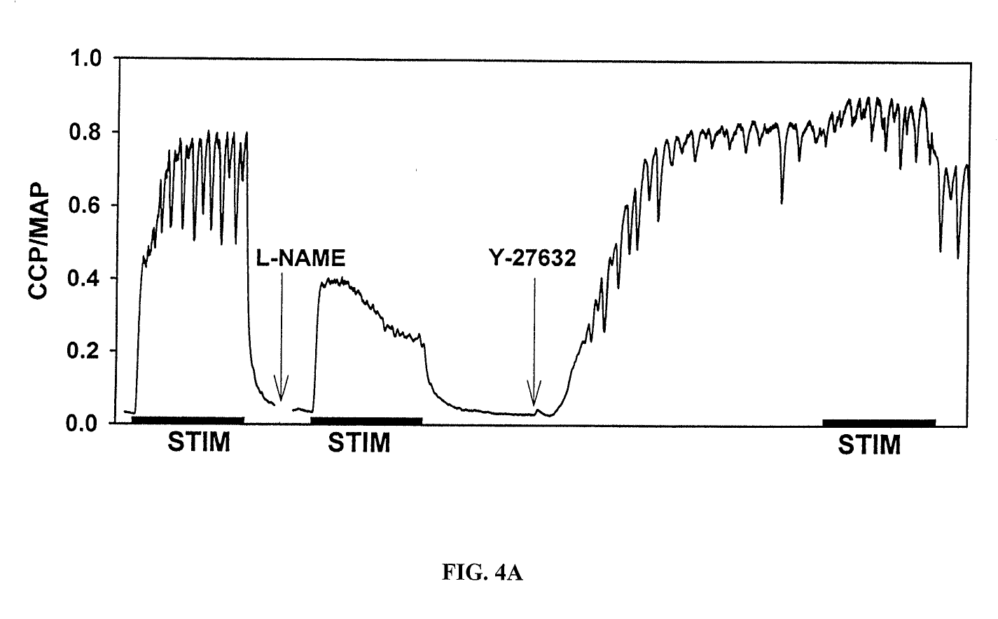

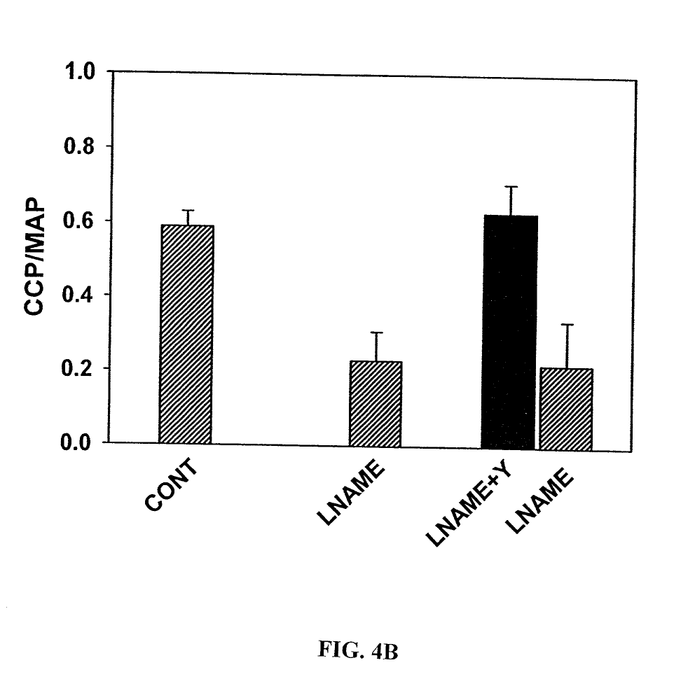

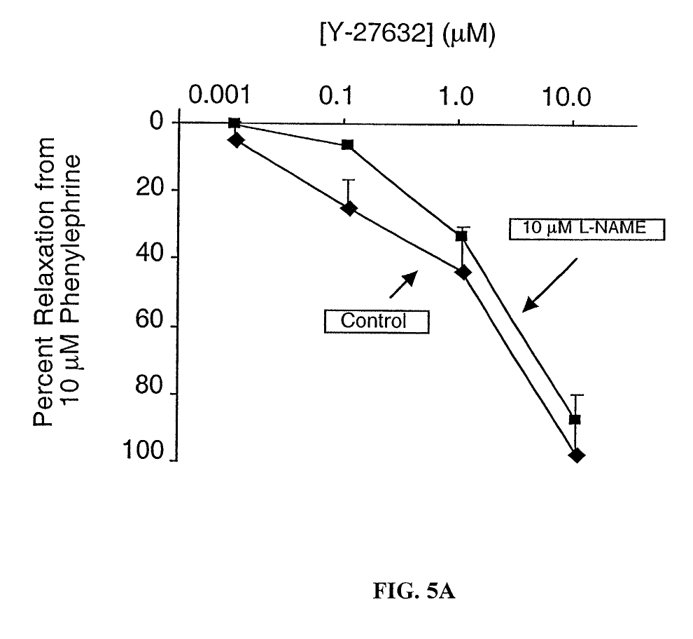

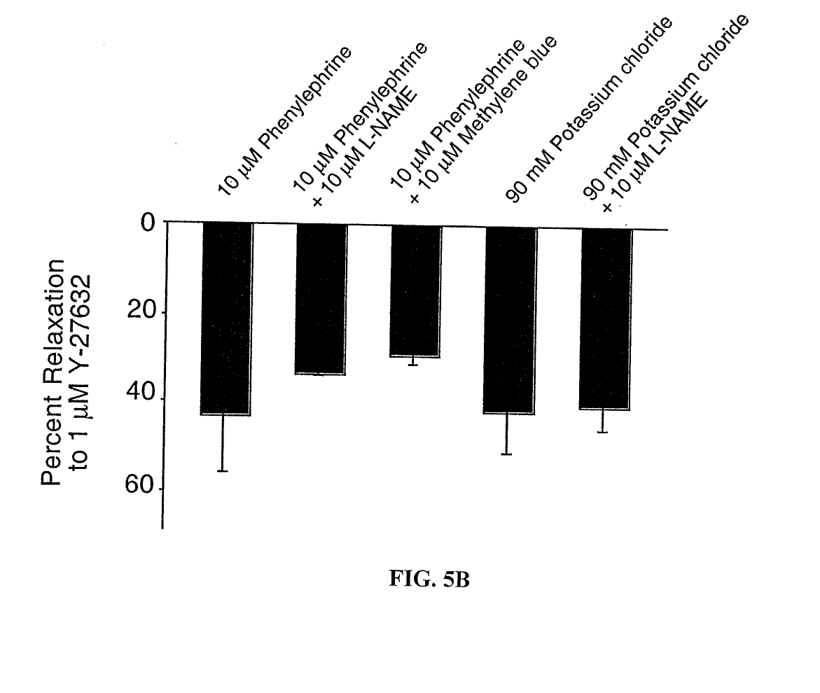

Also, and referring now to FIGS. 4 and 5, in an embodiment, Y-27632 is able to overcome the effects of the nitric oxide synthase (NOS) inhibitors N^{ω}-nitro-L-arginine methyl ester (L-NAME) and N^{ω}-nitro-L-arginine (L-NNA) on ganglionic stimulated increases in CCP, indicating that Rho-kinase inhibitors are not dependent on NO-mediated pathways to promote erection. Similarly, Y-27632 is able to restore ganglionic-stimulated increase in CCP/MAP even in the presence of inhibitors of cGMP formation such as methylene blue (MB) and oxadiazolo quinoxalin (ODQ), indicating that Rho-kinase inhibitors are effective in cases where the cGMP mediated increase in CCP/MAP is not effective (FIG. 5).

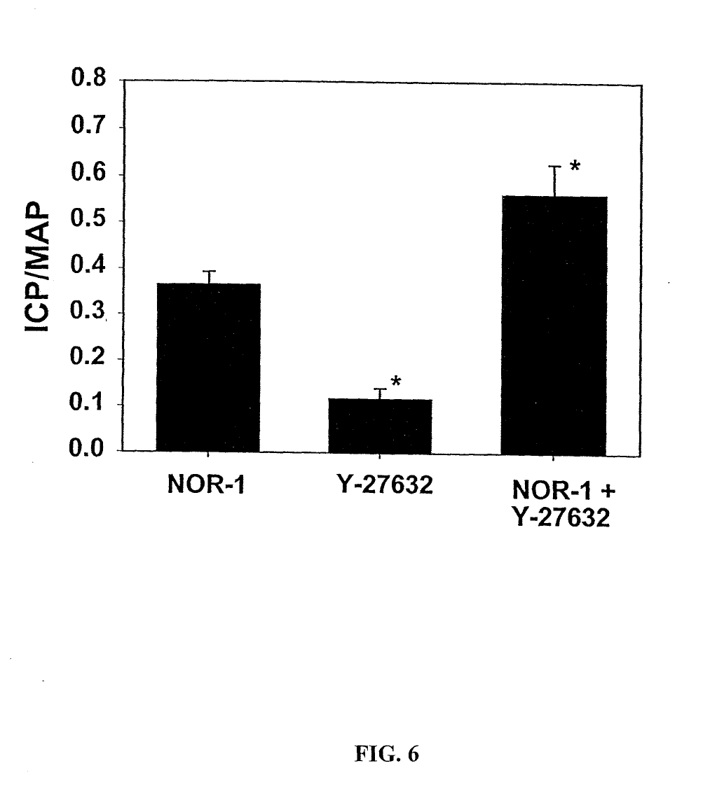

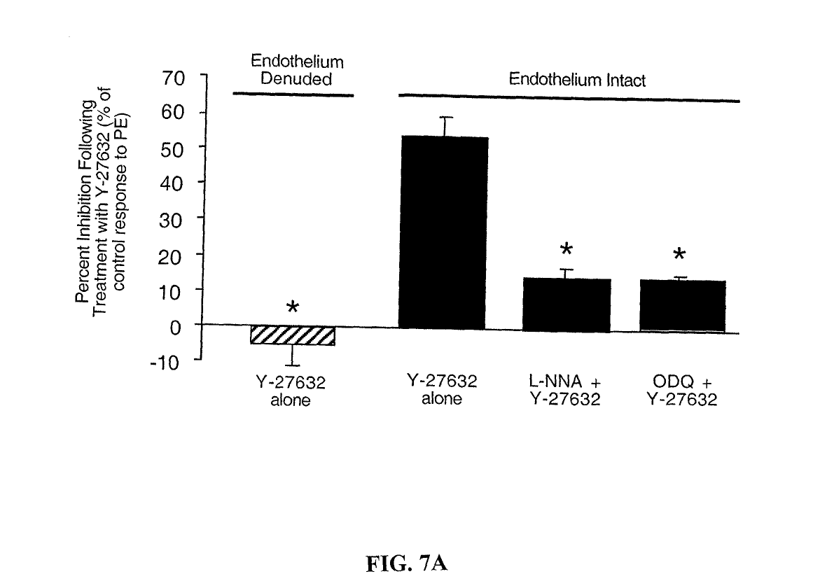

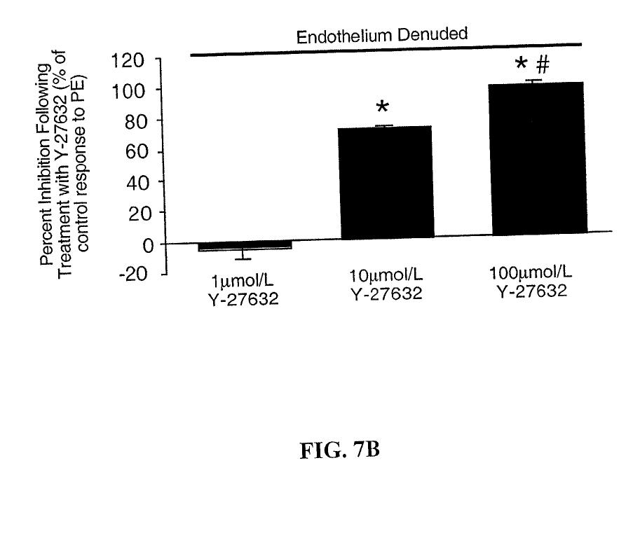

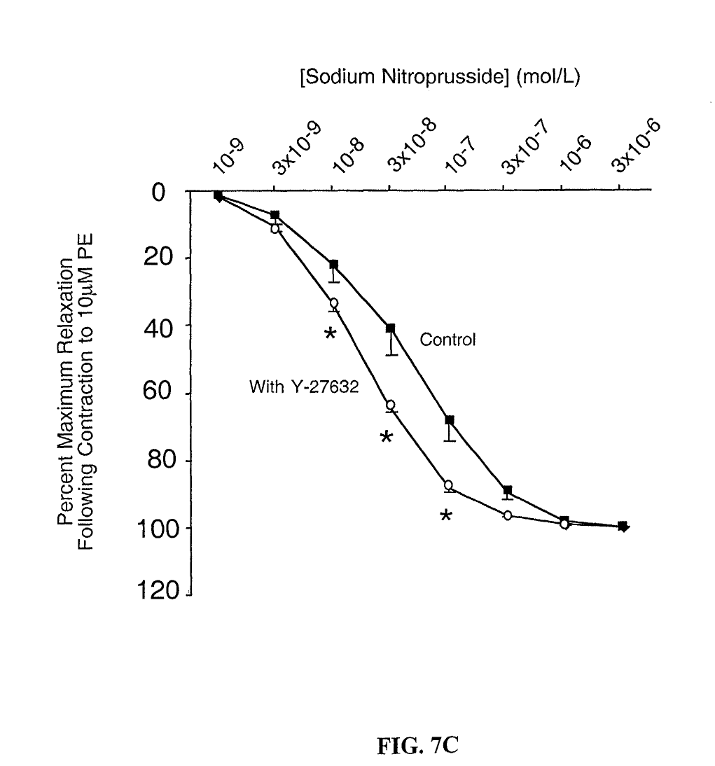

In some situations, however, and referring now to FIG. 6, erection induced by compounds comprising Rho-kinase inhibitors, such as Y-27632, may be additive to the effects of NO mediated erection which are brought about by NO donor compounds such as (+/−)-(E)-methyl-2-((E)-hydroxyimino)-5-nitro-6-methoxy-3-hexenamide (NOR-1), and the like. Thus, the effects of Y-27632, although not dependent on NO, may be increased by the addition of compounds that increase local NO concentration. For example, and referring now to FIG. 7, the effects of Y-27632 are more potent in vascular tissue which has endothelial cells and the ability to generate NO (endothelium intact) than in vascular tissue which has been stripped of endothelial cells and thus, cannot make NO (endothelium denuded). Thus, 1 μmol/L Y-27632 is effective in reversing phenylephrine (PE) induced contraction in tissue which can make NO (endothelium intact) but requires concentrations of up to 10 to 100 μmol/L Y-27632 for similar effectiveness in tissue that cannot produce NO (endothelium denuded) (FIGS. 7A and 7B). In addition, Rho-kinase inhibitors may be additive to the effects of NO or NO donor compounds such as sodium nitroprusside (FIG. 7C). Thus, in an embodiment, the present invention comprises a composition comprising a Rho-kinase inhibitor in combination with a compound which potentiates the effects of NO.

Therapeutics

In one aspect, the invention is a kit comprising a compound which attenuates RhoA and/or Rho-kinase in an organ subject to sexual stimulation which is mixed with a pharmaceutically acceptable carrier and packaged in units suitable for dispensing as individual dosages.

The invention contemplates methods of administration which are well known in the art. In an embodiment, administration of the compound is via intracavernous injection. For example, U.S. Pat. No. 4,127,118 describes a method of treating male impotence by injecting into the penis an appropriate vasodialator, such as an adrenergic blocking agent or a smooth muscle relaxant, to stimulate and enhance an erection.

In another embodiment, administration of the compound is transurethral. For example, U.S. Pat. Nos. 6,903,181 and 5,242,391 describe an apparatus and methods for treating sexual dysfunction, and specifically priapism and Peyronie's disease, using transurethral administration of a vasoconstrictor or other compound.

In another embodiment, administration of the compound is topical. For example, U.S. Pat. No. 4,801,587 describes the application of an ointment consisting of the vasodilators papaverine, hydralazine, sodium nitroprusside, phenoxybenzamine or phentolamine and a carrier to assist absorption through the skin. Also, U.S. Pat. No. 5,256,652 describes an aqueous topical composition of a vasodilator such as papaverine and hydroxypropyl-β-cyclodextrin, and U.S. Pat. No. 5,059,603 describes topical administration of caffeine and nitroglycerine with a suitable penetration enhancement compound.

In yet another embodiment, administration of the compound is oral or as an aerosol. Thus, U.S. Pat. Nos. 6,166,061, 6,124,337, 6,100,286, 6,051,594 and 5,981,563 describe methods for modulating the sexual response by oral administration of phentolamine. Also, U.S. Pat. No. 6,087,362 describes the treatment of sexual dysfunction by an oral regimen of apomorphine and sildenafil, and U.S. Pat. No. 6,007,824 describes treatment of sexual dysfunction using an oral dosage of L-arginine, ginseng and Zizyphi fructus.

In another embodiment, administration of the compound is sublingual. For example, U.S. Pat. No. 5,888,534 describes the sublingual administration of water-soluble drugs, such as apomorphine for impotence. The composition for sublingual administration is described as consisting essentially of a water-soluble drug which is a member of the group consisting of apomorphine hydrochloride, albuterol sulfate, timolol maleate, verapamil hydrochoride and naloxone hydrochloride, an osmotic agent, a swellable hydrophilic carrier and a water dispersible polymer. Also, U.S. Pat. No. 5,945,117 describes sublingual administration of apomorphine to ameliorate sexual dysfunction in females.

In another embodiment, administration is by ionophoresis or electroporation, a method of administration used to transmit a drug through the skin by application of an electrical charge. Thus, U.S. Pat. No. 6,266,560 BI describes electroporation for delivery of a vasoactive or androgenic composition to the penis to treat erectile dysfunction.

In yet another embodiment, administration of the drug is transrectal, as by a suppository or the like. For example, JP 7048254 describes the use of a formulations containing tetrahydrobenz[cd]indole-6-carboxamide for treating sexual dysfunction where the formulation is presented as a pharmaceutically acceptable form including suppositories.

In another embodiment, somatic cell gene therapy techniques to overexpress regulators and inhibitors of the RhoA/Rho-kinase signaling pathway provide an approach to the management of erectile dysfunction. It has been suggested that the penis is ideally suited for somatic cell gene therapy since the organ is easily accessible, and cavernosal cells have a low turnover rate (Christ, G. J., et al., Int. J. Impotence Res., 10, 111-112, 1998). Administration of naked DNA encoding the inducible form of nitric oxide synthase (iNOS) was shown to improve erectile function in rats (Garban, H., et al., Biol. Reprod., 56, 954-963, 1997). Erectile function was also improved in aged rats treated with hSlo DNA which encoded human smooth muscle maxi K⁺ channels (Christ, G. J., et al., Am J. Physiol 275, H600-608, 1998; see also U.S. Pat. No. 6,239,117 B1, specifically incorporated herein by reference, describing gene therapy for erectile dysfunction and bladder dysfunction by introduction of a DNA that regulates smooth muscle tone). More recently, it has been shown that adenoviral transfer of the endothelial form of NOS (eNOS) is beneficial to erectile function in older animals (Champion, H. C., et al., Proc. Natl. Acad. Sci., USA, 96, 11648-11652, 1999, Bivalacqua, T. J., et al., Int. J. Impotence Res., 12, S8-S17, 2000). In addition, gene transfer and gene deletion experiments have been performed to evaluate how heme oxygenase-1 and pre-proendothelin-1 gene expression alters vascular function (Duckers, H. J., et al, Nature Med., 7, 693-698, 2001; Schott, E., et al., Am. J. Physiol., 272, H2385-H2393, 1997).

Gene transfer studies to modify the RhoA/Rho-kinase pathway would involve the use of adenoviral vectors similar to those used by Bivalacqua et al., (2000). Recombinant adenoviruses are designed to contain gene sequences which encode for proteins which enhance or inhibit RhoA activation, such as but not limited to, guanosine nucleotide exchange factors (GEF), guanosine nucleotide dissociation inhibitors (GDI), guanosine activating protein (GAP), or proteins that inhibit Rho-kinase activity. For somatic gene therapy of cavernosal tissue, the adenoviral preparation, suspended in saline, is injected into the cavernous sinuses of the subject of interest. Controls may be performed wherein subjects are injected with adenoviral vector encoding a detectable marker such as β-galactosidase (but no sequences related to the RhoA/Rho-kinase pathway) to determine the success of the viral transfer. After allowing for infection and transfection of the exogenous DNA, (i.e. about 7 to 21 days), subjects are tested for their erectile response (e.g. in response to ganglionic stimulation or injection of vasoactive drugs) using standard in vivo and in vitro assays described herein. Erectile dysfunction due to a variety of causes (hypogonadism, diabetes, hypertension) may be treated with the adenoviral vectors.

Pharmaceutical formulations can be prepared by procedures known in the art. For example, the compounds can be formulated with common excipients, diluents, or carriers, and formed into tablets, capsules, suspensions, powders, and the like. Examples of excipients, diluents, and carriers, that are suitable for such formulations include the following: fillers and extenders such as starch, sugars, mannitol, and silicic derivates; binding agents such as carboxymethyl cellulose and other cellulose derivatives, alginates, gelatin, and polyvinyl pyrrolidone; moisturizing agents such as glycerol; disintegrating agents such as agar, calcium carbonate, and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate, and solid polyethyl glycols.

The compounds can also be formulated as elixirs or solutions for convenient oral administration or as solutions appropriate for parenteral administration, for instance by intramuscular, subcutaneous or intravenous routes. Additionally, the compounds are well suited to formulation as sustained release dosage forms and the like. The formulations can be so constituted that they release the active ingredient only or preferably in a particular part of the intestinal tract, possibly over a period of time. The coatings, envelopes, and protective matrices may be made, for example, from polymeric substances or waxes.

The present invention is suitable for treating sexual dysfunction which arises due to a variety of causes. Thus, sexual dysfunction (in both men and women) may arise as a result of reduced hormonal levels, psychological reasons, or physiological factors. For example, hypertension is often associated with a high prevalence of erectile dysfunction, and the drugs used to treat hypertension may cause erectile dysfunction. Hypertension is characterized by increased peripheral vascular resistance along with reduced nitric oxide (NO) availability. The primary reason men stop taking medication for high blood pressure is the problem of impotence. Thus, in an embodiment, the present invention is used to treat sexual dysfuntion associated with hypertension, or treatment of hypertension. Diabetes and the associated vascular disease is a major cause of erectile dysfunction. Thus, in an embodiment, the present invention is used to treat sexual dysfuntion associated with diabetes.

The present invention is also suitable for treating erectile dysfunction resulting from pelvic surgery. Men who undergo radical prostatectomy or other invasive pelvic surgical procedures may suffer from partial or complete loss of erectile function due to damage to elements of the nervous system associated with the erectile response. In addition, pelvic surgery may damage the arterial supply of the erectile tissue. Furthermore, veno-occlusive disorders leading to erectile dysfunction may result from smooth muscle damage or fibrosis. Thus, in an embodiment, the present invention is used to treat sexual dysfuntion associated with pelvic surgery or vascular damage.

In addition, the present invention is suitable for treating sexual dysfunction which results reduced hormonal levels. For example, in severely hypogonadal men, erectile dysfunction occurs. Similarly, any decline in circulating levels of gonadal hormones associated with age or loss of ovarian function may contribute to sexual dysfunction in females.

The present invention also provides the ability to treat diseases wherein the sexual dysfunction comprises an overly active erectile response, as for example the disorder of priapism. Thus, in one aspect, the present invention comprises a method to treat priapism in a patient, comprising increasing the activity of Rho-kinase in an organ subject to sexual stimulation in the patient. In priapism, sexual tissue is sustained for extended periods in an erect state. These prolonged periods of sustained erection and associated hypoxia in the erectile tissue may result in damage to the tissue and surrounding blood vessels including fibrosis and loss of vascular reactivity. By employing a means to transitorily increase the activity of Rho-kinase in sexual tissue, the tendency of the tissue to be maintained in an engorged state can be counteracted. Such methods could include treatment with RhoA or Rho-kinase agonists or other agents which activate elements of the RhoA/Rho-kinase pathway or the use of gene therapy to overexpress endogenous Rho-kinase activity in cases of chronic priapism.

Thus, the present invention relies on the discovery that inhibition vasoconstriction stimulates rat penile erection, and provides a candidate pathway for the development of agents suitable for the regulation of vasocongestive events such as penile erection and clitoral erection. In an embodiment, the invention employs agents which inhibit Rho-kinase. Inhibition of Rho-kinase stimulates an increase in intracavernosal pressure (ICP or CCP) and the erectile response independently of the effects of NO mediated erection. In addition, the lack of change in MAP upon administration of relevant doses of the Rho-kinase inhibitor illustrates the usefulness of Rho-kinase inhibitors in the treatment of erectile dysfunction. Features and advantages of the inventive concept covered by the present invention are illustrated in the following examples.

### EXAMPLES

Materials and Methods

The following materials and methods were utilized in the examples described herein. Y-27632 was a gift from Mitsubishi Pharma Corporation (Osaka, Japan). Indomethacin L-NNA, L-NAME, MB, ODQ and PE were purchased from Sigma.

For immunoblot and in vitro analysis, male Sprague-Dawley rats (200-250 g Harlan, Indianapolis) were anesthetized with sodium pentobarbital (50 mg/kg) intraperitoneal (IP), and cavernosal tissue removed as described. For in vivo experiments, male Holtzman rats (400 g, Harlan) were anesthetized with ketamine (87 mg/kg body weight) and xylazine (13 mg/kg body weight) and maintained on supplemental ketamine as needed. All rats were housed according to guidelines at the Medical College of Georgia.

Results are expressed as mean±s.e.m., and analyzed statistically with ANOVA followed by Newman posthoc analysis. Statistical significance was set at P<0.05.

### Example 1

Using immunoblot analysis with goat polyclonal antibody against ROCK-2 (anti-Rho-kinase), it was determined that endogenous Rho-kinase is present in rat cavernosal tissue (data not shown). Cavernosal strips (cleaned of the corpus spongiosum and dorsal vein), were snap frozen in liquid nitrogen and homogenized in cold radio-immunoprecipitation buffer [50 mM Tris-HCl (pH 7.4), 1.0% NP-40, 0.25% Na-deoxycholate, 150 mM NaCl, 1 mM EDTA, 1 mM phenylmethylsulfonyl fluoride (PMSF), 1 μg/mf aprotinin, 1 μg/ml leupeptin, 1 μg/ml pepstatin, 1 mM Na₃VO₄ and 1 mM NaF]. Samples were centrifuged (10,000 g, 4° C., 10 min), and the supernatant collected for protein quantification as described by Lowry, O. H., et al. (J. Biol. Chem. 193, 265-275, 1951). For immunoblot analysis, equal amounts of protein (50 μg per lane; rat brain extract as a positive control) were loaded and resolved by 10% sodium dodecyl sulfate polyacrylamide gel electrophoresis (SDS-PAGE). Protein was transferred to a nitrocellulose membrane (Immobilon-P, Millipore, Bedford, Mass.) using a Bio-Rad Mini-Protean III apparatus (100 V, 1 h, 4° C.) in the presence of 25 mM Trizma Base, 191.8 mM glycine and 20% methanol. The nitrocellulose membrane was then incubated with 5% skimmed milk in phosphate buffered saline (PBS) (30 min at 22° C.). After blocking, the membrane was incubated overnight (4° C.) with primary goat polyclonal antibody against Rho-A (SC-418) or Rho-kinase (ROCK-2) (both antibodies were obtained from Santa Cruz Biotech, Santa Cruz, Calif.), and subsequently incubated for 1.5 h with a horseradish peroxidase-linked secondary antibody against goat (1:15,000 dilution, 22° C., Jackson Labs, Bar Harbor, Me.). Antibody-bound protein was visualized using an enhanced chemiluminescence kit (Amersham, Piscataway, N.J.).

Both RhoA and Rho-kinase (ROCK-2) were detected in homogenates from isolated rat cavernosal tissue. RhoA protein corresponded to a band of about 23 kDa, whereas Rho-kinase-protein expression corresponded to a band of about 180 kDa.

### Example 2

Using an in vivo rat model, the effect of Rho-kinase inhibition on corpus cavernosum (CCP) and mean arterial pressure (MAP) was examined. The left carotid artery and right corpus cavernosum of the rat penis were cannulated for continuous monitoring of MAP and CCP respectively as described by Mills, T., et al., (Biol. Reprod. 59, 1413-1418, 1998; J. Appl. Physiol., 91: 1269-1273, 2001). Rats were anesthetized with intramuscular ketamine (87 mg/kg body wt) plus xylazine (13 mg/kg) and maintained on supplemental ketamine as needed. Vasoactive drugs were administered through a cannula in the left corpus cavernosum. For experiments measuring voltage-stimulated changes in CCP and MAP, stainless steel bipolar electrodes were positioned on the right major pelvic ganglion. To examine the effects of Y-27632 on ganglionic-induced changes in CCP/MAP, the nerve was stimulated with various voltages (0, 1, 2, 3, 4 and 5 V: 5-msec pulses at a frequency of 12 Hz) following intracavernosal injection of saline. Subsequently, Y-27632 (2.0, 20, 50, 200 and 400 nmol/kg, each rat receiving one dose) was administered intracavernosally, and the voltage stimulation series (0-5 V) was repeated. All pressure data were collected for analysis using Polyview data-acquisition software.

Injection of the specific Rho-kinase inhibitor, Y-27632 (FIG. 1) (Uehata et al., Nature 389, 990-994 (1997); Ishizaki, T., et al., Mol. Pharmacol., 57, 976-983 (2000)) (2.0-400 nmol/kg body weight in 2 μl saline), into the left corpus cavernosum sinuses resulted in a significant dose-dependent increase in CCP/MAP, reaching a plateau by about four minutes (FIG. 3A). The half-life of the effects of the Y-28632-induced (50 nmol/kg) increase in CCP/MAP was approximately 15 minutes. At the highest administered treatment of Y-27632 (400 nmol/kg), there was a significant decrease in MAP (−23.8±4.3 mm Hg, n=4), indicative of systemic Rho-kinase inhibition at this dose. In support of studies in normotensive rats (Uehata et al., Nature, 389, 990-994 (1997)), MAP was not significantly affected by treatment with lower doses of Y-27632 [change in MAP (mm Hg) upon administration of Y-27632 (2.0-200 nmol/kg), mean±S.E.M.: −3.5±1.8, −8.8±3.8, −9.8±2.5, −14.6±5.6, respectively; n=at least 4 per dose]. Thus, over the range of 2.0-200 nmol/kg of Y-27632, there was an increase in CCP/MAP to a maximum of 74% over control, independent of any significant changes in MAP. The ability of Y-27632 to increase the intracavernosal pressure independent of voltage stimulation indicates the presence of a significant basal Rho-kinase activity in the cavernosal smooth muscle.

The in vivo effects of Y-27632 on voltage-induced (i.e. nitric-oxide-mediated) increases in CCP/MAP are shown in FIG. 3B. Stimulation of the major pelvic ganglion (controlling cavernosal blood flow) resulted in a voltage-dependent increase in CCP/MAP, in accordance with previous findings (FIG. 3B, solid bars) (Dai, Y., et al., Am. J. Physiol., 279, R25-30, 2000). Administration of 200 nmol/kg Y-27632 into the cavernous sinuses potentiated the CCP/MAP response to ganglionic stimulation at each voltage (FIG. 3B, open bars). Moreover, administration of 200 nmol/kg Y-27632 increased the ganglionic-stimulated rise in CCP/MAP to near maximal levels even at the lowest stimulation voltages (FIG. 3B). Treatment with various doses of Y-27632 (2.0-200 nmol/kg), also potentiated ganglionic-stimulated increases in CCP/MAP at 5 V (21-38% increase in CCP/MAP over the range of Y-27632 tested).

### Example 3

The effect of the Rho-kinase inhibitor Y-27632 in the presence of nitric oxide synthase (NOS) inhibitors, N^{ω}-nitro-L-arginine (L-NNA, 200 μg/kg) and N^{ω}-nitro-L-arginine methyl ester (L-NAME, 200 ug/kg) is shown in FIGS. 4 and 5. To examine the effects of Y-27632 on ganglionic stimulated-CCP/MAP in the presence of NOS inhibition, a 5-V stimulus, previously determined to result in a maximal increase in CCP/MAP (Dai, Y., et al., Am. J. Physiol., 279, R25-30 (2000)) was delivered. After initial measurements of CCP and MAP during ganglionic stimulation, rats were treated with L-NNA, L-NAME (200 μg/kg body weight), or saline control, and after 5 min ganglionic stimulation and CCP/MAP measurements were repeated. Rats were subsequently administered either Y-27632 (50 nmol/kg) or saline. After 5 min, a 5-V stimulation was repeated, and CCP and MAP measurements were recorded.

It was found that administration of L-NNA or L-NAME into the cavernosal sinuses did not alter CCP or MAP from baseline. As previously shown (Reilly, C. M., et al., J. Andrology 18, 110-115, 1997; Reilly, C. M., et al., J. Andrology 18, 588-594, 1997; Moody, J. A., et al., J. Urology 158, 942-947, 1997). L-NAME treatment significantly attenuated the increase in CCP/MAP induced by a 5 V stimulation of the major pelvic ganglion (FIG. 4A: tracing from one animal; and 4B: four separate experiments). The attenuating effect of NOS inhibition on the ganglionic-induced CCP/MAP increase was overcome, however, by administration of Y-27632 (50 nmol/kg) (FIG. 4A and 4B). In FIG. 4B the second L-NAME bar indicates that rats receiving L-NAME remained suppressed over the time period during which Y-27632 was administered (i.e. L-NAME+no treatment). Thus, these results indicate that Y-27262 can act independently of NO-induced vasodilation.

### Example 4

Rho-kinase inhibition can also overcome muscle contraction due to inhibition of cyclic GMP formation. Inhibition of cyclic GMP formation results in a reduced ganglionic-stimulated rise CCP/MAP (Reilly et al., J. Andrology 18, 588-594 (1997). To examine the effects of Y-27632 on ganglionic stimulated-CCP/MAP in the presence of guanylate cyclase inhibition, a 5-V stimulus, previously determined to result in a maximal increase in CCP/MAP (Dai, Y., et al., Am. J. Physiol., 279, R25-30 (2000)) was delivered. After initial measurements of CCP and MAP during ganglionic stimulation, rats were treated with MB or ODQ (300-500 μg/kg), or saline control, and after 5 min ganglionic stimulation and CCP/MAP measurements were repeated. Rats were subsequently administered either Y-27632 (50 nmol/kg) or saline. After 5 min, a 5-V stimulation was repeated, and CCP and MAP measurements were recorded.

It was found that the intra-cavernosal administration of the guanylate cyclase inhibitors methylene blue (MB, 300-500 μg/kg) or 1H-[1,2,4]oxadiozolo-[4,3-a]quinoxalin-1 (ODQ, 300-500 μg/kg) resulted in a significant inhibition of the ganglionic-stimulated rise in CCP/MAP at 5 V [CCP/MAP (mean±s.e.m.): control 0.68±0.2 (n=4), MB 0.49±0.04 (n=4), P<0.05; control 0.78±0.04, ODQ 0.50±0.07 (n=4), P<0.05]. However, treatment with 50-200 nmol/kg Y-27632 even in the continued presence of MB or ODQ significantly restored the ganglionic-stimulated increase in CCP/MAP [CCP/MAP (mean±s.e.m.): MB+Y-27632: 0.73±0.03 (n=4), ODQ+Y-27632: 0.80±0.04 (n=4)] (Table 2).

TABLE 2

| | CCP/MAP at 5 V | | CCP/MAP at 5 V |
|---|---|---|---|
| Treatment | (mean ± s.e.m.) | Treatment | (mean ± s.e.m.) |
| Control | 0.68 ± 0.2 | Control | 0.78 ± 0.04 |
| MB | 0.49 ± 0.04 | ODQ | 0.50 ± 0.07 |
| MB + Y-27632 | 0.73 ± 0.03 | ODQ + Y-27632 | 0.80 ± 0.04 |

Using in vitro analysis of isolated cavernosal tissue, the effects of Y-27632 on agonist-induced contraction in the presence and absence of NOS/guanylate cyclase inhibition was examined. Cavernosal strips (3-4 mm long) were prepared by removal of the corpus spongiosum and dorsal vein, and the proximal portion of the corpus cavernosum divided across the midline of the intact penis. Right and left longitudinal strips were mounted in an isolated muscle bath for measurement of contractile force generation. Strips were bathed in physiologic salt solution (130 mM NaCl, 4.7 mM KCl, 1.18 mM KHPO₄, 1.17 mM Mg₂SO₄, 1.6 mM CaCl₂-2H₂O, 14.9 mM NaHCO₃, 5.5 mM dextrose, and 0.03 mM CaNa₂ EDTA), at 37° C., and gassed with 95% O₂/5% CO₂. Cavernosal strips were contracted with 10 μM phenylephrine (PE) (pre-determined to cause near maximal force generation), and subsequently relaxed with Y-27632. To examine the effects of Y-27632 on non-receptor mediated pre-contraction of rat cavernosal strips, isolated tissue was contracted with potassium chloride (KCl) (90 mM) and subsequently treated with 1 μM Y-27632 (IC₅₀ value from PE pre-contraction). Additional strips (pre-contracted with PE or KCl) were treated for 15 min with 10 μM L-NAME (to inhibit NOS) or 10 μM MB (to inhibit guanylate cyclase, see below) before the addition of Y-27632.

It was found that treatment of cavernosal strips with Y-27632 resulted in dose-dependent relaxation of the PE-induced tone (IC₅₀=1 μM), in agreement with findings from other vascular smooth muscle (Uehata, M. et al., Nature, 389, 990-994 (1997), Weber et al., Pharmacology, 63, 129-133, 2001) (FIG. 5A). Subsequent treatment of strips with L-NAME (10 μM, 15 min) had no effect on the PE-induced contraction. Similar to the in vivo findings discussed above, incubation with 10 μM L-NAME or 10 μM MB did not significantly alter the relaxation response of cavernosal strips in 1 μM Y-27632 (FIG. 5B). The addition of 1 μM Y-27632 to the muscle bath resulted in a similar extent of relaxation of cavernosal strips pre-constricted with KCl (in the presence or absence of NOS inhibition) or PE (FIG. 5B) in contrast to studies reporting an attenuated effect of Y-27632 on the relaxation of conduit vessels pre-constricted with KCl as opposed to PE (Uehata, M. et al., Nature, 389, 990-994 (1997); Weber, D. S. et al., Pharmacology, 63, 129-133, 2001). These results (Examples 3 and 4) indicate that NOS and guanylate cyclase inhibition do not significantly alter the effect of Rho-kinase inhibition in the rat penile tissue suggesting that Y-27632-induced cavernosal relaxation and rise in CCP/MAP is not dependent on NO.

### Example 5

Although Y-27632-induced cavernosal relaxation and rise in CCP/MAP is not dependent on nitric oxide (NO), NO may inhibit RhoA/Rho-kinase induced vasoconstriction as part of the normal erectile response. Studies by Sauzeau et al., and others have demonstrated that activation of the NO/cGMP/cGMP dependent protein kinase (CKG) pathway leads to inhibition of RhoA and smooth muscle relaxation. In these studies recombinant RhoA was phosphorylated by cGK at Ser-188, resulting in the inhibition of RhoA promotion of stress fiber formation (Sauzeau, V., et al., J. Biol. Chem., 275, 21722-21729, 2000; Sawada, N., et al., Biochem., Biophys. Res. Comm., 280, 798-805, 2001). In addition, sodium nitroprusside (SNP) and constitutively active cGK were demonstrated to inhibit the phenylephrine (PE) or lysophophatidic acid-induced translocation of RhoA from the cytosolic to membrane fraction in rat aorta and NIH3T3 cells, respectively. Thus, there appears to be two possible mechanisms by which NO-induced increases in cGK could control RhoA activity: cGK may inhibit RhoA translocation to the membrane, and/or cGK may directly phosphorylate, and thereby inhibit, RhoA activity.

FIG. 6 shows that inhibition of Rho-kinase with Y-27632 enhances the erectile response resulting from the NO donor drug, (+/−)-(E)-methyl-2-((E)-hydroxyimino)-5-nitro-6-methoxy-3-hexenamide (NOR-1) (Biomol Research Laboratories, Inc., Plymouth, Pa.). While continuously recording ICP and MAP, a small dose of NOR-1 was injected directly into the erectile tissue and an intermediate erectile response (measured as and increase in ICP/MAP) occurred (NOR-1). After a recovery period, a low dose of the Rho-kinase inhibitor Y-27632 was injected (Y-27632), which was then followed (after a subsequent recovery period) by another NOR-1 injection (NOR-1+Y-27632). It can be seen that both agents cause an erectile response (measured as an increase in ICP/MAP) and that in combination the magnitude of the response is additive (FIG. 6).

FIG. 7 shows the results of an experiment using rat aorta strips in which a substantial portion of the endothelial cells have been removed (endothelial layer denuded). Endothelial cells are the source of NO in vascular tissue, thus removal of the endothelium removes any source of NO release for this in vitro system. It was found that aorta strips with endothelium (endothelium intact and therefore NO⁺) are more sensitive to the Rho-kinase inhibitor than the strips without endothelium (endothelium denuded and therefore NO⁻). Additionally, in the endothelium intact strips (NO⁺) the effect of Y-27632 was decreased in the presence of inhibitors of nitric oxide synthase or guanylate cyclase.

In these experiments, aorta (3 mm rings) were cleaned of adherent connective tissue and hung on stainless steel hooks attached between a force transducer and stationary mount for the measurement of isometric force generation. Endothelium was removed from some vessels by gentle rubbing of the lumen with a steel wire. Aortic rings were then placed in an isolated chamber, bathed in physiologic salt solution (mmol/L: 130 NaCl, 4.7 KCl, 1.18 KHPO₄, 1.17 MgSO₄, 1.6 CaCl₂-2H₂O, 14.9 NaHCO₃, 5.5 dextrose, and 0.03 CaNa₂EDTA; 37° C.) and gassed with 95% O₂/5%CO₂ Rings were set at 3 g passive tension and treated with indomethacin (1 μmol/L) to inhibit prostaglandin synthesis. Following a one hour equilibration period, vessels were contracted with phenylephrine (PE) (0.1 μmol/L) and subsequently treated with acetylcholine (1 μmol/L) to test for the presence of endothelium.

To examine the effect of endothelium removal on the response to Rho-kinase inhibition, endothelium intact (NO⁺) and denuded (NO⁻) aortic rings were contracted with 10 μmol/L phenylephrine (PE) and subsequently treated with Y-27632. Treatment with 1 μmol/L Y-27632 resulted in a significant inhibition of force generation to 10 μmol/L PE in endothelium intact rate aortic rings, but was completely ineffective in endothelium denuded vessels (FIG. 7A). Additionally, in the presence of N^{ω}-nitro-L-arginine (L-NNA) (100 μmol/L) or 1H-[1,2,4]oxadiozolo-[4,3-a]quinoxalin-1 (ODQ) (10 μmol/L) (to inhibit NO synthase or guanylate cyclase, respectively), 1 μmol/L Y-27632 was significantly less effective at inhibiting the contractile response to 10 μmol/L PE, suggesting that endogenous NO/cGMP signaling functions can decrease Rho-kinase activity (FIG. 7A).

In endothelium denuded rings, 10 μmol/L or 100 μmol/L Y-27632 resulted in a concentration dependent inhibition of the contractile response to 10 μmol/L PE (FIG. 7B) indicative of the ability of higher concentrations Rho-kinase inhibitors to act independently of NO. Also, treatment with the NO-donor, sodium nitroprusside (SNP) (30 nmol/L), restored the ability of 1 umol/L Y27632 to inhibit the response to PE (10 μmol/L) in endothelium denuded rings, demonstrating that exogenous NO donors can be used to mediate Rho-kinase signaling (data not shown).

Finally, to examine the effect of Rho-kinase inhibition on the relaxation response to NO, endothelium-denuded vessels were first contracted with 10 μmol/L PE (in the presence or absence of 1 μmol/L Y-27632, and subsequently relaxed with increasing concentrations of the NO donor, SNP. Vessels treated with Y-27632 prior to PE-induced contraction exhibited an increased vasodialator sensitivity to SNP as compared to untreated rings (FIG. 7C). This suggests that inhibition of Rho-kinase can be employed to add to, or potentiate, NO-induced muscle relaxation.

### Example 6

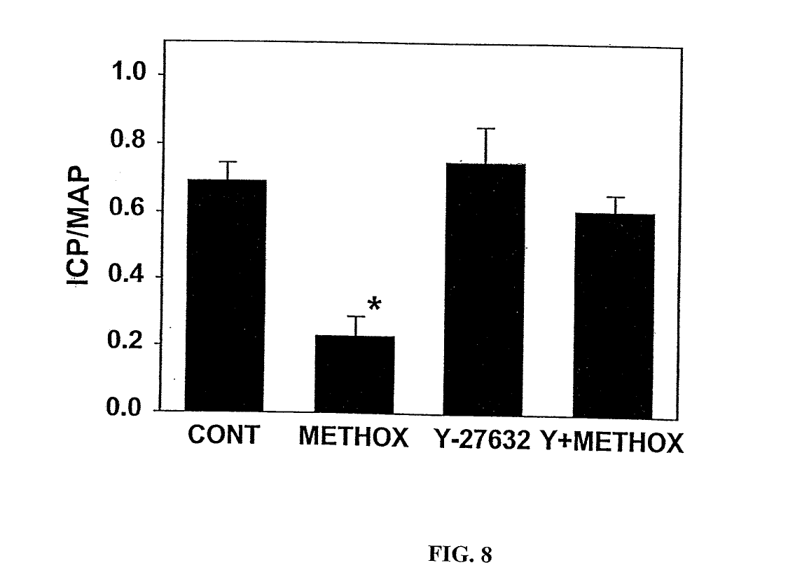

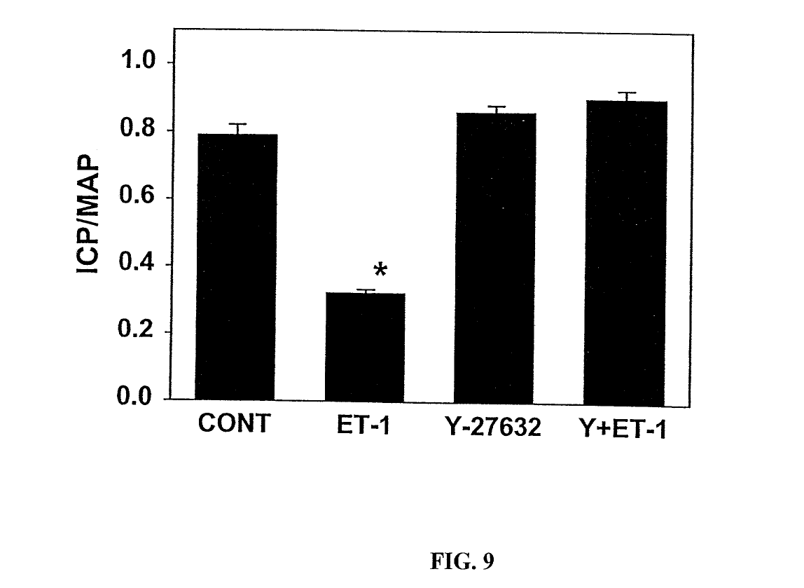

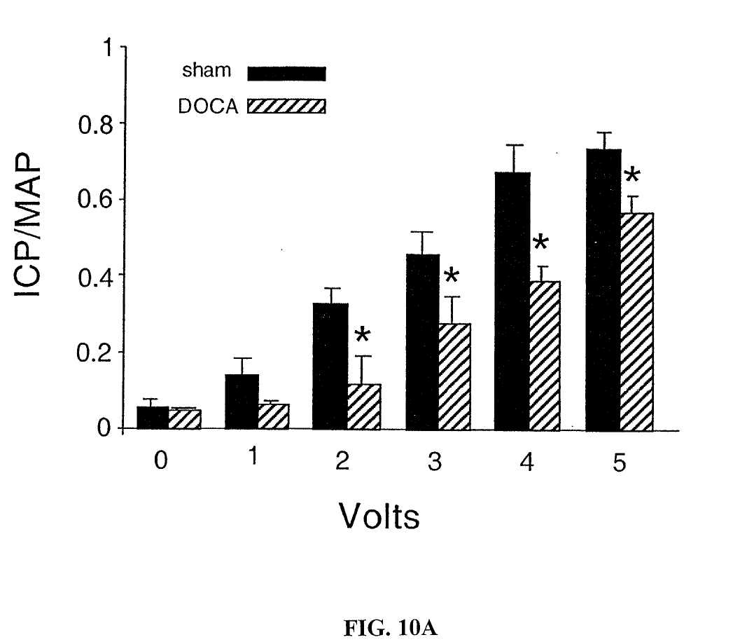

This example shows that vasoconstrictors which act in the penis act via the Rho-kinase pathway. Thus, FIGS. 8 and 9 show results where after an initial (5V) ganglionic stimulation (Cont), either the α-adrenergic agonist, Methoxamine (METHOX) (10 μg/kg) or endothelin-1 (ET-1) (50 pmol) was injected into the cavernous sinuses. Both METHOX (FIG. 8) and ET-1 (FIG. 9) exerted a potent vasoconstrictor action and lowered the erectile response to 5V ganglionic stimulation. If, however, Y-27632 (50 nmol) was administered before the vasoconstrictor (Y+METHOX) (FIG. 8) or (Y+ET-1) (FIG. 9), the vasoconstrictor effect was prevented.

### Example 7

This example shows that the erectile response is significantly reduced in two animal models of hypertension and that inhibition of Rho-kinase activity in these animals with Y-27632 restores erectile function. The two animal models were deoxycorticosterone-salt induced hypertension (DOCA-salt) rats and spontaneously hypertensive rats which are stroke prone (SHRSP).

In these experiments, male Sprague-Dawley rats (200-250 g; Harlan) were made DOCA-salt hypertensive by uninephrectomy and implantation of a deoxycorticosterone-acetate (DOCA) silastic pellet at the back of the neck. DOCA rats received saltwater (1% NaCl, 0.2% KCl) for 4 weeks. Sham control rats were uninephrectomized and drank tap water. Male stroke prone-spontaneously hypertensive rats (12 weeks old, 350 g) were obtained from the breeding colony at the Medical College of Georgia, Augusta, Ga. Systolic blood pressures were taken by tail cuff measurement (DOCA/sham: after 4 weeks treatment; SHRSP: at 12 weeks of age). Measurement of ICP/MAP was as described above.

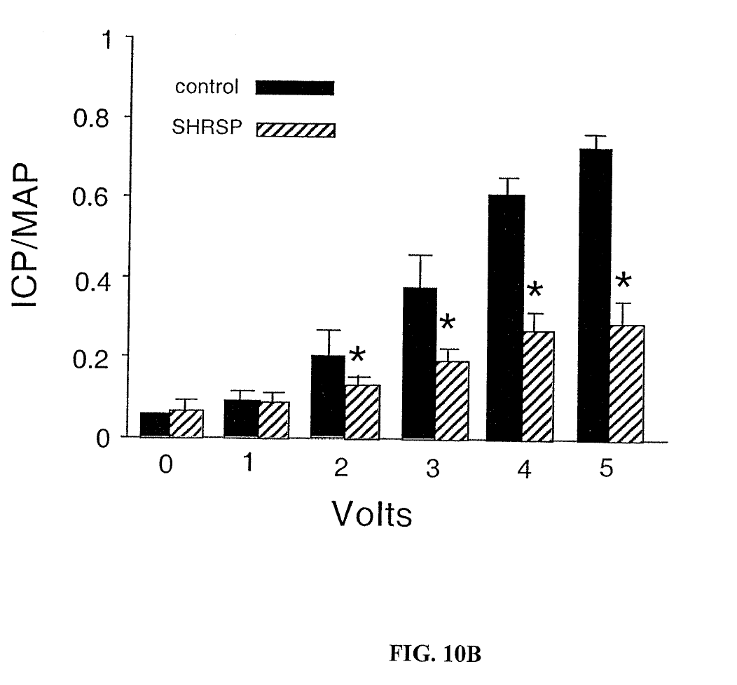

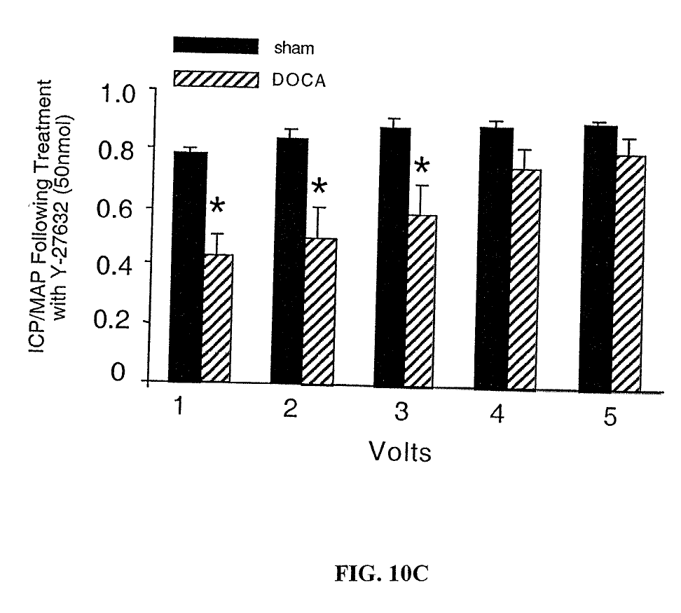

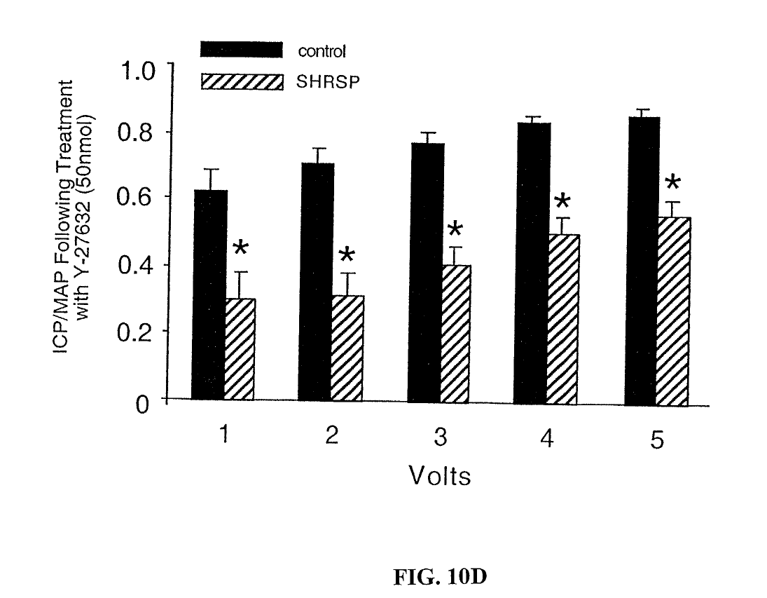

For measurement of ganglionic-mediated increases in ICP/MAP, the major pelvic ganglion was stimulated over a range of voltages (1-5 V). Stimulation resulted in a voltage-dependent increase in ICP/MCP, with the increase in ICP/MAP decreased in the hypertensive DOCA and SHRSP rats as compared to sham-treated or normotensive controls, repectively (FIG. 10A and B, respectively). To examine the effect of Y-27632 on the voltage induced increase in ICP/MAP, Y-27632 (50 nmol/kg) was injected into the left corpus cavernosum and the increased ICP response allowed to plateau. Subsequently, the ICP/MAP response to ganglionic stimulation was assessed over a range of voltages. It was found that Y-27632 resulted in an increase in ICP/MAP, thereby elevating the erectile response to near normal levels in DOCA rats and significantly increasing the erectile response in SHRSP rats (FIGS. 10C and D, respectively).

### Example 8

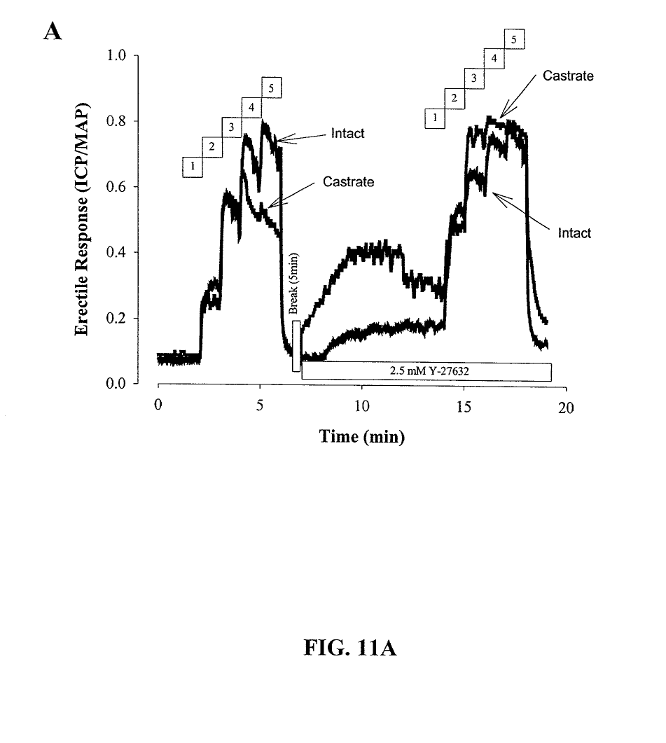

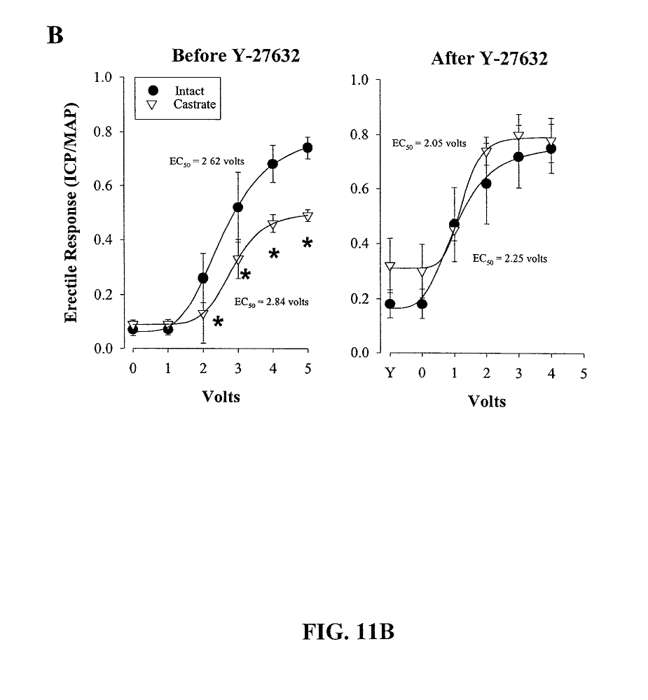

Rats which have been rendered severely hypogonadal by surgical castration show a diminished erectile response. When castrated rats that display an impaired erectile response are treated with Y-27632, the erectile response is restored to levels similar to those in age-matched intact animals (FIG. 11). FIG. 11A shows representative traces of the erectile response to graded stimulation (1-5 V, as indicated above the tracing) of the major pelvic ganglion before and after intracavernosal injection of the Rho-kinase inhibitor Y-27632. FIG. 11B shows the erectile responses for each of the applied voltages (based on the value for 2 minutes of stimulation at a given voltage) for several (N=5-6) animals.

In addition, it was found that the increased contractile sensitivity to the adrenergic agonist phenylephrine (PE) associated with castration was overcome by introduction of Y-27632, with an EC50 for Y-27632 of about 3 μM. This suggests that erectile dysfunction seen with a hypogonadal state is associated with augmented vasoconstrictor activity that can be reversed with the use of an inhibitor of Rho-kinase and restoring the erectile response to normal.

Finally, western blots revealed that in castrated animals, RhoA and Rho-kinase protein levels were elevated. In these experiments, protein homogenates from the corpus cavernosum of rats 10 days after castration were compared to protein levels from age matched intact rats. It was found that there was an approximately 16 fold increase in RhoA protein, and a 2 fold increase in Rho-kinase in the castrated animals indicating up-regulation of the RhoA/Rho-kinase pathway with castration.

Publications referred to throughout the text of this document are incorporated by reference in their entireties in order to more fully describe the state of the art as known to those skilled therein as of the date of the invention described and claimed herein.

What is claimed is:

1. A method for treating male or female sexual dysfunction which comprises administering a composition comprising a compound which attenuates RhoA and/or Rho-kinase activity in an organ subject to sexual stimulation and a pharmaceutically acceptable carrier to an individual in need of such treatment.

2. The method of claim 1 wherein said composition comprises a compound that inhibits the activity of Rho-kinase enzyme in an organ subject to sexual stimulation.

3. The method of claim 2 wherein said compound comprises the structure of formula (I) or a functional derivative thereof, 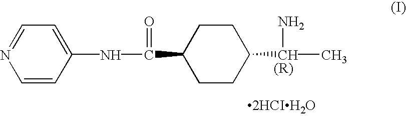
wherein a functional derivative comprises a compound which can inhibit the activity of Rho-kinase mediated phosphorylation and thereby increase intracavernosal blood pressure (ICP) relative to mean arterial pressure (MAP).

4. The method of claim 2 wherein said compound that inhibits the activity of Rho-kinase enzyme is administered in a dose ranging from 2.0 to 400 nmol/kg body weight.

5. The method of claim 2 wherein said compound that inhibits the activity of Rho-kinase enzyme is administered in a dose ranging from 5.0 to 200 nmol/kg body weight.

6. The method of claim 2 wherein said compound that inhibits the activity of Rho-kinase enzyme is administered in a dose ranging from 40 to 100 nmol/kg body weight.

7. The method of claim 1 wherein said composition comprises a compound that reduces the amount of active Rho-kinase enzyme.

8. The method of claim 7 further comprising a compound that inhibits RhoA activity.

9. The method of claim 8 further comprising a compound that inhibits binding of GTP to RhoA enzyme.

10. The method of claim 8 further comprising a compound that inhibits translocation of RhoA enzyme to the cellular membrane.

11. The method of claim 8 wherein said composition comprises an inhibitor of Rho-kinase and a second compound which potentiates the effects of nitric oxide.

12. The method of claim 1 wherein said composition comprises a compound that acts on a downstream target of Rho-kinase such as myosin light chain phosphatase, calponin, myosin light chain, CPI-17, and others.

13. The method of claim 1 further comprising intracavernous administration of said composition.

14. The method of claim 1 further comprising topical administration of said composition.

15. The method of claim 1 further comprising oral administration of said composition.

16. The method of claim 1 further comprising sublingual administration of said composition.

17. The method of claim 1 further comprising nasal administration of said composition.

18. The method of claim 1 further comprising transurethral administration of said composition.

19. The method of claim 1 further comprising transrectal administration of said composition.

20. The method of claim 1 further comprising ionophoresis or electroporation for administration of said composition.

21. The method of claim 1 further comprising gene therapy to alter various proteins in the RhoA/Rho-kinase signal transduction pathway.

22. The method of claim 1 wherein the sexual dysfunction comprises sexual dysfunction associated with hypogonadism.

23. The method of claim 22 wherein said hypogonadism is associated with reduced levels of androgen hormones.

24. The method of claim 1 wherein the sexual dysfunction comprises sexual dysfunction associated with hypertension, diabetes, or pelvic surgery.

25. The method of claim 1 wherein the sexual dysfunction comprises sexual dysfunction associated with treatment of certain drugs such as those to treat hypertension, depression or anxiety.

26. A method to treat priapism in a patient comprising increasing the activity of the RhoA/Rho-kinase pathway in an organ subject to sexual stimulation in said patient.

27. A composition for treating male or female sexual dysfunction comprising a compound which attenuates RhoA and/or Rho kinase activity in an organ subject to sexual stimulation and a pharmaceutically acceptable carrier.

28. The composition of claim 27 further comprising at least one compound that inhibits the activity of Rho-kinase enzyme in an organ subject to sexual stimulation.

29. The composition of claim 28 further comprising a compound comprising the structure of formula (I) or a functional derivative thereof, 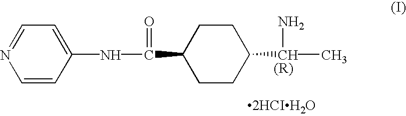
wherein a functional derivative comprises a compound which can inhibit the activity of Rho-kinase mediated phosphorylation and thereby increase intracavernosal pressure (ICP) relative to mean arterial pressure (MAP).

30. The composition of claim 28 wherein said compound that inhibits the activity of Rho-kinase enzyme is administered in a dose ranging from 2.0 to 400 nmol/kg body weight.

31. The composition of claim 28 wherein said compound that inhibits the activity of Rho-kinase enzyme is administered in a dose ranging from 5.0 to 200 nmol/kg body weight.

32. The composition of claim 28 wherein said compound that inhibits the activity of Rho-kinase enzyme is administered in a dose ranging from 40 to 100 nmol/kg body weight.

33. The composition of claim 27 wherein said composition comprises a compound that reduces the amount of active Rho-kinase enzyme.

34. The composition of claim 33 wherein said composition comprises a compound that inhibits RhoA activity.

35. The composition of claim 34 wherein said composition comprises a compound that inhibits translocation of RhoA enzyme to the cellular membrane.

36. The composition of claim 34 wherein said composition comprises at least one compound that that inhibits binding of GTP to RhoA enzyme.

37. The composition of claim 34 wherein said composition comprises an inhibitor of Rho-kinase and a second compound which potentiates the effects of nitric oxide.

38. The composition of claim 27 wherein said composition comprises a compound that acts on a downstream target of Rho-kinase, such as myosin light chain phosphatase, calponin, myosin light chain, CPI-17, and others.

39. The composition of claim 27 wherein said composition is suitable for intracavernous administration.

40. The composition of claim 27 wherein said composition is suitable for topical administration.

41. The composition of claim 27 wherein said composition is suitable for oral administration.

42. The composition of claim 27 wherein said composition is suitable for sublingual administration.

43. The composition of claim 27 wherein said composition is suitable for nasal administration.

44. The composition of claim 27 wherein said composition is suitable for transurethral administration.

45. The composition of claim 27 wherein said composition is suitable for transrectal administration.

46. The composition of claim 27 wherein said composition is suitable for administration by ionophoresis or electroporation.

47. A kit for treating male or female sexual dysfunction comprising at least one compound which attenuates RhoA and/or Rho-kinase activity in an organ subject to sexual stimulation and a pharmaceutically acceptable carrier.

48. The kit of claim 47 further comprising aliquots packaged in units suitable for dispensing as individual dosages.

49. The kit of claim 47 wherein said compound inhibits Rho-kinase activity.

50. The kit of claim 49 wherein said compound comprises the structure of formula (I) or a functional derivative thereof, 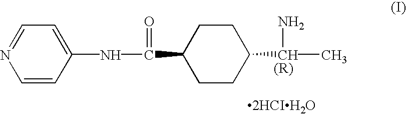
wherein a functional derivative comprises a compound which can inhibit the activity of Rho-kinase mediated phosphorylation and thereby increase intracavernosal blood pressure (ICP) relative to mean arterial pressure (MAP).
